SEQUENCE LISTING (54 sequences)

SEQ ID NO: 1 - protein, 21 aa, Synthetic construct
KKNQKKSVKLSKKALKLAKKN

SEQ ID NO: 2 - protein, 21 aa, Synthetic construct
KKLLKKWVKLSKKALKLAQSL

SEQ ID NO: 3 - protein, 21 aa, Synthetic construct
  SITE 1: note = Acetylation of N-terminal lysine
KKLLKKWVKLSKKALKLAQSL

SEQ ID NO: 4 - protein, 21 aa, Synthetic construct
  SITE 1: note = Acetylation of N-terminal lysine
KKLLKKWVKLSKKALKLAQSL

SEQ ID NO: 5 - protein, 20 aa, Synthetic construct
KLLKKWVKLSKKALKLAQSL

SEQ ID NO: 6 - protein, 19 aa, Synthetic construct
LLKKWVKLSKKALKLAQSL

SEQ ID NO: 7 - protein, 18 aa, Synthetic construct
LKKWVKLSKKALKLAQSL

SEQ ID NO: 8 - protein, 17 aa, Synthetic construct
KKWVKLSKKALKLAQSL

SEQ ID NO: 9 - protein, 16 aa, Synthetic construct
KWVKLSKKALKLAQSL

SEQ ID NO: 10 - protein, 15 aa, Synthetic construct
WVKLSKKALKLAQSL

SEQ ID NO: 11 - protein, 20 aa, Synthetic construct
KKLLKKWVKLSKKALKLAQS

SEQ ID NO: 12 - protein, 19 aa, Synthetic construct
KKLLKKWVKLSKKALKLAQ

SEQ ID NO: 13 - protein, 18 aa, Synthetic construct
KKLLKKWVKLSKKALKLA

SEQ ID NO: 14 - protein, 17 aa, Synthetic construct
KKLLKKWVKLSKKALKL

SEQ ID NO: 15 - protein, 16 aa, Synthetic construct
KKLLKKWVKLSKKALK

SEQ ID NO: 16 - protein, 15 aa, Synthetic construct
KKLLKKWVKLSKKAL

SEQ ID NO: 17 - protein, 21 aa, Synthetic construct
KKLLKKLVKLSKKALKLAQSL

SEQ ID NO: 18 - protein, 21 aa, Synthetic construct
KKLLKKKVKLSKKALKLAQSL

SEQ ID NO: 19 - protein, 21 aa, Synthetic construct
KKLLKKWLKLSKKALKLAQSL

SEQ ID NO: 20 - protein, 21 aa, Synthetic construct
KKLLKKWKKLSKKALKLAQSL

SEQ ID NO: 21 - protein, 21 aa, Synthetic construct
KKLLKKWVKLLKKALKLAQSL

SEQ ID NO: 22 - protein, 21 aa, Synthetic construct
KKLLKKWVKLKKKALKLAQSL

SEQ ID NO: 23 - protein, 21 aa, Synthetic construct
KKLLKKWVKLSKKLLKLAQSL

SEQ ID NO: 24 - protein, 21 aa, Synthetic construct
KKLLKKWVKLSKKKLKLAQSL

SEQ ID NO: 25 - protein, 21 aa, Synthetic construct
KKLLKKWVKLSKKALKLLQSL

SEQ ID NO: 26 - protein, 21 aa, Synthetic construct
KKLLKKWVKLSKKALKLKQSL

SEQ ID NO: 27 - protein, 21 aa, Synthetic construct
KKLLKKWVKLSKKALKLALSL

SEQ ID NO: 28 - protein, 21 aa, Synthetic construct
KKLLKKWVKLSKKALKLAKSL

SEQ ID NO: 29 - protein, 21 aa, Synthetic construct
KKLLKKWVKLSKKALKLAQLL

SEQ ID NO: 30 - protein, 21 aa, Synthetic construct
KKLLKKWVKLSKKALKLAQKL

SEQ ID NO: 31 - protein, 21 aa, Synthetic construct
  VARIANT 14: note = X is 2-aminoisobutyric acid
KKLLKKWLKLSKKXLKLALSL

SEQ ID NO: 32 - protein, 21 aa, Synthetic construct
  VARIANT 18: note = X is 2-aminoisobutyric acid
KKLLKKWLKLSKKALKLXLSL

SEQ ID NO: 33 - protein, 21 aa, Synthetic construct
  VARIANT 14: note = X is 2-aminoisobutyric acid
  VARIANT 18: note = X is 2-aminoisobutyric acid
KKLLKKWLKLSKKXLKLXLSL

SEQ ID NO: 34 - protein, 21 aa, Synthetic construct
KKLLKKLLKLSKKALKLALSL

SEQ ID NO: 35 - protein, 21 aa, Synthetic construct
  VARIANT 7: note = X is 4-biphenyl-L-alalnine
KKLLKKXLKLSKKALKLALSL

SEQ ID NO: 36 - protein, 21 aa, Synthetic construct
  VARIANT 7: note = X is 1-naphthyl-L-alanine
KKLLKKXLKLSKKALKLALSL

SEQ ID NO: 37 - protein, 21 aa, Synthetic construct
  VARIANT 7: note = X is 2-naphthyl-L-alanine
KKLLKKXLKLSKKALKLALSL

SEQ ID NO: 38 - protein, 21 aa, Synthetic construct
  SITE 1: note = d-lysine
  SITE 2: note = d-lysine
  SITE 3: note = d-leucine
  SITE 4: note = d-leucine
  SITE 5: note = d-lysine
  SITE 6: note = d-lysine
  SITE 7: note = d-tryptophan
  SITE 8: note = d-leucine
  SITE 9: note = d-lysine
  SITE 10: note = d-leucine
  SITE 11: note = d-serine
  SITE 12: note = d-lysine
  SITE 13: note = d-lysine
  SITE 14: note = d-alanine
  SITE 15: note = d-leucine
  SITE 16: note = d-lysine
  SITE 17: note = d-leucine
  SITE 18: note = d-alanine
  SITE 19: note = d-leucine
  SITE 20: note = d-serine
  SITE 21: note = d-leucine
KKLLKKWLKLSKKALKLALSL

SEQ ID NO: 39 - protein, 21 aa, Synthetic construct
KKLLKKWLKLSKKALKLALSL

SEQ ID NO: 40 - protein, 20 aa, Synthetic construct
KLLKKWLKLSKKALKLALSL

SEQ ID NO: 41 - protein, 19 aa, Synthetic construct
LLKKWLKLSKKALKLALSL

SEQ ID NO: 42 - protein, 18 aa, Synthetic construct
LKKWLKLSKKALKLALSL

SEQ ID NO: 43 - protein, 20 aa, Synthetic construct
KKLLKKWLKLSKKALKLALS

SEQ ID NO: 44 - protein, 19 aa, Synthetic construct
KKLLKKWLKLSKKALKLAL

SEQ ID NO: 45 - protein, 18 aa, Synthetic construct
KKLLKKWLKLSKKALKLA

SEQ ID NO: 46 - protein, 19 aa, Synthetic construct
  SITE 1: note = d-lysine
  SITE 2: note = d-lysine
  SITE 3: note = d-leucine
  SITE 4: note = d-leucine
  SITE 5: note = d-lysine
  SITE 6: note = d-lysine
  SITE 7: note = d-tryptophan
  SITE 8: note = d-leucine
  SITE 9: note = d-lysine
  SITE 10: note = d-leucine
  SITE 11: note = d-serine
  SITE 12: note = d-lysine
  SITE 13: note = d-lysine
  SITE 14: note = d-alanine
  SITE 15: note = d-leucine
  SITE 16: note = d-lysine
  SITE 17: note = d-leucine
  SITE 18: note = d-alanine
  SITE 19: note = d-leucine
KKLLKKWLKLSKKALKLAL

SEQ ID NO: 47 - protein, 19 aa, Synthetic construct
  VARIANT 7: note = X is 2-naphthyl-L-alanine
KKLLKKXLKLSKKALKLAL

SEQ ID NO: 48 - protein, 19 aa, Synthetic construct
  SITE 1: note = d-lysine
  SITE 2: note = d-lysine
  SITE 3: note = d-leucine
  SITE 4: note = d-leucine
  SITE 5: note = d-lysine
  SITE 6: note = d-lysine
  VARIANT 7: note = X is 2-naphthyl-D-alanine
  SITE 8: note = d-leucine
  SITE 9: note = d-lysine
  SITE 10: note = d-leucine
  SITE 11: note = d-serine
  SITE 12: note = d-lysine
  SITE 13: note = d-lysine
  SITE 14: note = d-alanine
  SITE 15: note = d-leucine
  SITE 16: note = d-lysine
  SITE 17: note = d-leucine
  SITE 18: note = d-alanine
  SITE 19: note = d-leucine
KKLLKKXLKLSKKALKLAL

SEQ ID NO: 49 - protein, 15 aa, Synthetic construct
  VARIANT 7: note = X is S, W, L, K, 4-biphenyl-L-alalnine, 1-naphthyl-L-alanine, or 2-naphthyl-L-alanine
  VARIANT 8: note = X is V, L, or K
  VARIANT 11: note = X is S, L, or K
  VARIANT 14: note = X is A, L, K, or 2-aminoisobutyric acid
KKLLKKXXKLXKKXL

SEQ ID NO: 50 - protein, 15 aa, Synthetic construct
  VARIANT 1: note = X is S, W, L, K, 4-biphenyl-L-alalnine, 1-naphthyl-L-alanine, or 2-naphthyl-L-alanine
  VARIANT 2: note = X is V, L, or K
  VARIANT 5: note = X is S, L, or K
  VARIANT 8: note = X is A, L, K, or 2-Aminoisobutyric acid
  VARIANT 12: note = X is A, L, K, or 2-Aminoisobutyric acid
  VARIANT 13: note = X is Q, L, or K
  VARIANT 14: note = X is S, L, or K
XXKLXKKXLKLXXXL

SEQ ID NO: 51 - protein, 21 aa, Synthetic construct
  VARIANT 7: note = X is S, W, L, K, 4-biphenyl-L-alalnine, 1-naphthyl-L-alanine, or 2-naphthyl-L-alanine
  VARIANT 8: note = X is V, L, or K
  VARIANT 11: note = X is S, L, or K
  VARIANT 14: note = X is A, L, K, or 2-aminoisobutyric acid
  VARIANT 18: note = X is A, L, K, or 2-aminoisobutyric acid
  VARIANT 19: note = X is Q, L, or K
  VARIANT 20: note = X is S, L, or K
KKLLKKXXKLXKKXLKLXXXL

SEQ ID NO: 52 - protein, 434 aa, Synthetic construct
MAMQKIFAREILDSRGNPTVEVDLHTAKGRFRAAVPSGASTGIYEALELRDGDKGRYLGKGVLKAVENINNTLGPALLQKKLSVVDQEKVDKFMIELDGTENKSKFGANAILGVSLAVCKAGAAEKGVPLYRHIADLAGNPDLILPVPAFNVINGGSHAGNKLAMQEFMILPVGASSFKEAMRIGAEVYHHLKGVIKAKYGKDATNVGDEGGFAPNILENNEALELLKTAIQAAGYPDKVVIGMDVAASEFYRNGKYDLDFKSPDDPARHITGEKLGELYKSFIKNYPVVSIEDPFDQDDWATWTSFLSGVNIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAIFAGRKFRNPKAK

SEQ ID NO: 53 - protein, 21 aa, Synthetic construct
KVNQIGSVTESIQACKLAQSN

SEQ ID NO: 54 - protein, 22 aa, Synthetic construct
GIGKFLKKAKKFGKAFVKILKK

(19) United States
(12) Patent Application Publication
(10) Pub. No.: US 2026/0327263 A1
(43) Pub. Date: Oct. 22, 2026

(54) Anti-Bacterial Peptides

(71) Applicant: AGENCY FOR SCIENCE, TECHNOLOGY AND RESEARCH, Singapore (SG)

(72) Inventors: Smarajit Chakraborty, Singapore (SG); Sugii Shigeki, Singapore (SG); Cheng San Brian Chia, Singapore (SG)

(21) Appl. No.: 19/482,409

(22) PCT Filed: May 8, 2024
(86) PCT No.: PCT/SG2024/050299

§ 371 Date: Nov. 7, 2025

(30) Foreign Application Priority Data
May 8, 2023 (SG) 10202301275X

(51) Int. Cl. C07K 7/08; A61K 38/00; A61P 31/04

(52) U.S. Cl. CPC C07K 7/08; A61P 31/04; A61K 38/00

(57) ABSTRACT

This technology relates to anti-bacterial peptides and a method of treating infections using the peptides.

### CROSS-REFERENCE TO RELATED APPLICATIONS

This application claims priority to 10202301275X, filed on 8 May 2023, which is incorporated herein by reference in its entirety.

### FIELD OF THE INVENTION

The present invention relates generally to the field of cell biology. In particular, the present invention relates to antimicrobial peptides.

### BACKGROUND

Chronic wound management is a large and growing problem due to the aging population, and the sharp rise in the incidence of diabetes and obesity worldwide. The incidence of chronic wounds such as diabetic foot ulcers and burn injuries has been increasing over years. In Singapore, for example, the number of diabetic individuals is estimated to rise to 1 million by 2050 and the population of Singaporeans aged above 65 will reach up to 900,000 by 2030 (Gov.sg). Specifically with regards to diabetic foot ulceration, for example, Singapore has one of the highest rates of lower extremity amputations as compared to other developed countries (Committee of Supply Debate 2016, Minister of Health Gan Kim Yong, 13 Apr. 2016), which poses economical burdens to the healthcare system and individuals. Therefore, the problem of treatment of chronic wounds calls for effective treatment options.

Chronic wounds are typically due to the occurrence of nosocomial bacterial infection that complicates skin lesions. In some cases, pathogens, necrotic tissue, and the extracellular matrix (ECM) secreted by the pathogens form into a membranous layer termed “biofilm”. Biofilms are difficult to manage due to the presence of the protective matrix barrier. As of today, antibiotics stand as the first line of treatment against wound infections, for example, in the form of wound dressings to facilitate tissue regeneration. However, none of them actively manage chronic infections beyond the use of limited choices of existing antibiotics. Furthermore, bacteria that cause chronic wounds are predominantly multidrug resistant (MDR) bacteria, which can easily survive standard antibiotic treatments by gaining antimicrobial resistance (AMR). The emergence of antibiotic resistant bacterial infections accompanied with the dearth of new antibiotics creates challenges to the management of chronic wounds. Attempts have been made to treat chronic wounds infected with multidrug resistant (MDR) bacterial strains, such as the development of combination therapies using antibiotics in combination with either anti-microbial peptides (AMPs) or polymers. Unfortunately, most of those therapies resulted in adverse effects, further increased in antimicrobial resistance (AMR), and high cost in treatment, which are not desirable for patients and healthcare facilities. Alternative methods such as skin grafting are considered as gold standard in chronic wound management. However, skin grafting is limited by the quality and quantity of donor skin, which does not solve the problem of bacterial clearance either. Similarly, other new procedures such as delivery of angiogenesis-inducing growth factors and cytokines and hyperbaric oxygen therapy do not resolve the fundamental issue of infections. As an alternative, anti-microbial peptides (AMPs) are at the limelight due to the less likelihood to induce bacterial resistance. Yet most of the anti-microbial peptides (AMPs) failed at preclinical stages due to the following reasons: (i) limited broad-spectrum anti-microbial activity (works only on certain types of bacteria); (ii) targeting of eukaryotic cells, (iii) hemolytic and/or cytotoxic effects at anti-microbial concentrations; (iv) low anti-microbial activity in clinically relevant environments; and (v) proteolytic degradation. Regarding the targeting of eukaryotic cells, such targeting with substances can carry certain risks due to cytotoxic effects. Such targeting substances can damage or kill cells, affecting not just targeted cells but also surrounding healthy cells, leading to broad cellular harm. Additionally, targeting of eukaryotic cells raises concerns about acute toxicity, which results from short-term exposure and can vary from mild to severe symptoms, and systemic toxicity, which impacts multiple organ systems, causing widespread health issues. Such toxicity risks are significant for both topical applications, applied directly to the skin and systemic applications, meant to affect the body more broadly.

Therefore, what is needed is an anti-microbial agent effective in treating chronic wounds, and method of treating chronic wounds using the anti-microbial agent. Furthermore, other desirable features and characteristics will become apparent from the subsequent detailed description and the appended claims, taken in conjunction with the accompanying drawings and this background of the disclosure.

### SUMMARY

In one aspect, the present disclosure provides a peptide comprising the sequence of KKLLKKX₁X₂KLX₃KKX₄L (SEQ ID NO: 49), wherein: X₁ is S, W, L, K, 4-biphenyl-L-alalnine, 1-naphthyl-L-alanine, or 2-naphthyl-L-alanine; X₂ is V, L, or K; X₃ is S, L, or K; and X₄ is A, L, K, or 2-aminoisobutyric acid.

In another aspect, the present disclosure provides a peptide comprising the sequence of X₁X₂KLX₃KKX₄LKLX₅X₆X₇L (SEQ ID NO: 50), wherein: X₁ is S, W, L, K, 4-biphenyl-L-alalnine, 1-naphthyl-L-alanine, or 2-naphthyl-L-alanine; X₂ is V, L, or K; X₃ is S, L, or K; X₄ is A, L, K, or 2-aminoisobutyric acid; X₅ is A, L, K, or 2-aminoisobutyric acid; X₆ is Q, L, or K; and X₇ is S, L, or K.

In another aspect, the present disclosure provides a peptide comprising the sequence of KKLLKKX₁X₂KLX₃KKX₄LKLX₅X₆X₇L (SEQ ID NO: 51), wherein: X₁ is S, W, L, K, 4-biphenyl-L-alalnine, 1-naphthyl-L-alanine, or 2-naphthyl-L-alanine; X₂ is V, L, or K; X₃ is S, L, or K; X₄ is A, L, K, or 2-Aminoisobutyric acid; X₅ is A, L, K, or 2-Aminoisobutyric acid; X₆ is Q, L, or K; and X₇ is S, L, or K.

In another aspect, the present disclosure provides a composition comprising one or more of the peptides as disclosed herein.

In another aspect, the present disclosure provides a pharmaceutical composition comprising the peptide as disclosed herein, or the composition as disclosed herein, and a pharmaceutically acceptable carrier or excipient.

In another aspect, the present disclosure provides a wound dressing comprising the peptide as disclosed herein, the composition as disclosed herein, or the pharmaceutical composition as disclosed herein.

In another aspect, the present disclosure provides a nucleic acid composition encoding the peptide as disclosed herein.

In another aspect, the present disclosure provides an expression vector comprising the nucleic acid composition as disclosed herein.

In another aspect, the present disclosure provides a host cell comprising one or more of the nucleic acid compositions as disclosed herein, or one or more of the expression vectors as disclosed herein.

In another aspect, the present disclosure provides a method of treating or preventing the recurrence of a bacterial infection in a subject, comprises administering to the subject a pharmaceutically effective amount of the peptide as disclosed herein, the composition as disclosed herein, the pharmaceutical composition as disclosed herein, or the wound dressing as disclosed herein.

In another aspect, the present disclosure provides a method of promoting wound healing and tissue regeneration in a subject, wherein the method comprises administering to the subject a pharmaceutically effective amount of the peptide as disclosed herein, the composition as disclosed herein, the pharmaceutical composition as disclosed herein, or the wound dressing as disclosed herein.

In another aspect, the present disclosure provides a method of reducing biofilm/preventing formation of biofilm in a subject, wherein the method comprises administering to the subject a pharmaceutically effective amount of the peptide as disclosed herein, the composition as disclosed herein, the pharmaceutical composition as disclosed herein, or the wound dressing as disclosed herein.

### FIGURE DESCRIPTION

FIG. 1 provides a demonstration of the development process for peptide variants starting from the base sequence of E1A, which is a 21-residue short region within peptide ENO3 (SEQ ID NO: 52). The variants are generated by separating the distribution of basic amino acid (Lysine) and hydrophobic residues that align on opposing faces, forming a unique cationic arm which can interact with the negatively charged bacterial membrane, thereby disrupting the membrane of the bacteria. FIG. 1A shows a Helical Wheel of the peptide sequence of E1A and FIG. 1B shows a Helical Wheel of the exemplary peptide sequence of E1AM1. Helical Wheels predict protein sequence segments with either helical or non-helical potential by showing projections of a protein's amino acid sequence onto a plane perpendicular to the axis of the helix. The hydrophobic and the polar residues, charged and uncharged are indicated in different shades. Comparing FIG. 1A and FIG. 1B, the changes from E1A to E1AM1 are the separation of positively charged, basic Lysine residues from other non-polar or polar (acid or uncharged) residues and substitution of negatively charged Glutamate (10) to Leucine to prevent possible repulsion of the peptide in contact with bacterial membrane. FIG. 1C shows a Helical Wheel of the exemplary peptide sequence of E1AM2, which includes further substitutions such as Asparagine (3) to Leucine, Glutamine (4) to Leucine, Serine (7) to Tryptophan, Lysine (19) to Glutamine, Lysine (20) to Serine, and Asparagine (21) to Leucine from E1AM1 (FIG. 1B) to obtain more hydrophobic residues at the other end which can target the lipid chains of the bacterial membrane. FIG. 1D provides the full sequences of the peptide variants EIA, E1AM1, and E1AM2, with the changes in E1AM1 and E1AM2 compared to the E1A sequences indicated in bold.

FIG. 2 verifies the effects of exemplary variant peptides in disrupting bacterial membranes. To test whether exemplary peptide E1AM2 causes membrane damage in bacteria, live and dead staining of exemplary bacteria strain Pseudomonas aeruginosa (ATCC 14213, PA) are performed in the presence of different concentrations of E1AM2 peptide. As confirmed in the representative images of Pseudomonas aeruginosa (PA) in FIG. 2A, E1AM2 at its 1× minimum inhibitory concentration (MIC) (3.125 μM) and 1X minimum bactericidal concentration (MBC) (6.25 μM) caused membrane damage evident from the 100% incorporation of propidium iodide (PI) in all the cells. Scale bar-3 μm. The lesser number of PA cells observed upon E1AM2 treatment at these concentrations is due to leaking of the bacteria caused by membrane disruption. Thus, peptide variants with the cationic arm show bacterial membrane disrupting ability. FIG. 2B provides a quantification of Syto-9 and PI-positive Pseudomonas aeruginosa (PA) cells 24 hours post E1AM2 treatment. The reduction in positively stained cells shows the disruption of bacterial cells at increased concentrations of tested variant peptide.

FIG. 3 shows the effects of the exemplary peptide E1AM2 (SEQ ID NO: 2) in disintegration of biofilm. Scanning electron microscope (SEM) analysis is conducted for Pseudomonas aeruginosa (PA) in the presence or absence of E1AM2 (1X minimum inhibitory concentration (MIC)) using heat-inactivated Pseudomonas aeruginosa (PA) as a positive control. In the absence of E1AM2, Pseudomonas aeruginosa (PA) formed extracellular polysaccharides (EPS), confirming the presence of biofilm, as exemplified in the representative scanning electron microscope (SEM) images of FIG. 3. Scale bar-1 μm. Addition of E1AM2 completely disrupts the biofilm, showing no presence of extracellular polysaccharides (EPS), comparable to heat-inactivated Pseudomonas aeruginosa (PA). The variant peptides, such as E1AM2, have therefore been validated to show anti-biofilm activity, which is critical for bacterial clearance at the wound sites.

FIG. 4 shows that the exemplary variant peptide E1AM2 displays in vivo anti-bacterial efficacy. As demonstrated in a mouse excisional wound infection model caused by Pseudomonas aeruginosa (PA) infection, E1AM2 at 1x minimum bactericidal concentration (MBC) (6.25 μM) is effective in reducing PA infection in vivo, indicating significant bacterial clearance at the wound site in contrast to phosphate buffered saline (PBS) negative control. FIG. 4 provides the colony forming unit (CFU) counts of Pseudomonas aeruginosa (PA) normalized to volume and the weight of the tissue excised for counting for E1AM2 (6.25 μM, 1x minimum bactericidal concentration (MBC)) or PBS. The CFU counts drop significantly in the presence of exemplary peptide E1AM2, demonstrating the in vivo bactericidal effects of the peptide. Excisional wounds in mice are infected with Pseudomonas aeruginosa (PA) for 24 hrs followed by 24 hrs of treatment with E1AM2 or PBS. Each circle represents a single mouse. Statistical analysis was performed by the two-tailed t-test using GraphPad, *p<0.05.

FIG. 5 provides further alternation of the variant peptide sequences based on E1AM2 (SEQ ID NO: 2) to improve efficacy and cost-effectivity. Substitution of amino acids to either leucine or lysine residues in a state-of-the-art HPA3NT3-analog peptide has been shown to result in an increased anti-bacterial potency and reduced hemolytic activity. As shown in the Helical Wheels structures in FIG. 5A to FIG. 5V compared to E1AM2, different amino acids are replaced by either leucine or lysine in E1AM2 at positions, 7, 8, 11, 14, 18, 19, and 20 of the base sequence of EIA (KVNQIGSVTESIQACKLAQSN, SEQ ID NO: 53), all of which have intact cationic arm, which was previously shown to disrupt bacterial membrane. The minimum inhibitory concentration (MIC) and minimum bactericidal concentration (MBC) of the variant peptides are tested against exemplary bacteria Pseudomonas aeruginosa (PA) and methicillin resistant Staphylococcus aureus (BAA1717-USA 300, SA), with a positive control pexiganan (PEX), which is known for anti-bacterial effects against PA and SA (see Table 5). The tested variants show reduced or comparable MIC and MBC compared to E1AM2 and pexiganan against both PA and SA. For example, variants Q19L (SEQ ID NO: 27) (i.e. variant peptide carrying a mutation from amino acid Q to L at position 19 of base sequence E1A having SEQ ID NO: 53) and V8L (SEQ ID NO: 19) (i.e. variant peptide carrying a mutation from amino acid V to L at position 8 of base sequence E1A having SEQ ID NO: 53) exhibit reduced MIC and MBC while variant W7L (SEQ ID NO: 7) exhibits reduced MIC against PA. (W7L, SEQ ID NO: 17; W7K, SEQ ID NO: 18; V8L, SEQ ID NO: 19; V8K, SEQ ID NO: 20; SIIL, SEQ ID NO: 21; S11K, SEQ ID NO: 22; A14L, SEQ ID NO: 23; A14K, SEQ ID NO: 24; A18L, SEQ ID NO: 25; A18K, SEQ ID NO: 26; Q19L, SEQ ID NO: 27; Q19K, SEQ ID NO:28; S20L, SEQ ID NO: 29; S20K, SEQ ID NO: 30; W7LV8LQ19L, SEQ ID NO: 34; V8LQ19L, SEQ ID NO: 39; V8LQ19L N-1, SEQ ID NO: 40; V8LQ19L N-2, SEQ ID NO: 41, V8LQ19L N-3, SEQ ID NO: 42; V8LQ19L C-1, SEQ ID NO: 43; V8LQ19L C-2, 44; V8LQ19L C-3, SEQ ID NO: 45)

FIG. 6 demonstrates the long-term anti-bacterial effects of the exemplary variant peptides without development of antimicrobial resistance. The development of antimicrobial resistance is defined by greater than a 4-fold change to their initial minimum inhibitory concentration (MIC) at day 1. Exemplary bacteria strains Pseudomonas aeruginosa (PA) and Staphylococcus aureus (SA) are subjected to continuous daily serial passaging over several days, with treatment of exemplary variant peptides E1AM2 (SEQ ID NO: 2) and Q19L (SEQ ID NO: 39) at subinhibitory concentrations. Antibiotics such as cefepime, piperacillin, ciprofloxacin, gentamicin, colistin, and known bactericidal peptide pexiganan (SEQ ID NO: 54) are used as controls. The treatments were discontinued when the MIC increased by more than four-fold from the initial MIC or when MIC reached >100 μM. As shown in FIG. 6A, Pseudomonas aeruginosa (PA) developed resistance against all the five antibiotics as well as pexiganan. Exemplary peptide variants E1AM2 and Q19L prevented resistance acquisition in Pseudomonas aeruginosa (PA) throughout the entire course of treatment over 28 days. FIG. 6B shows that Q19L variant prevented resistance development in Staphylococcus aureus (SA). Both of the exemplary peptide variants E1AM2 and Q19L prevented occurrence of antimicrobial resistance during long-term treatment, better than all the antibiotics and state of the art antimicrobial peptide pexiganan.

FIG. 7 shows that the exemplary peptide variants E1AM2 (SEQ ID NO: 2) and Q19L (SEQ ID NO: 27) increase keratinocyte migration in 2D scratch wound model, indicating potentials in improving wound healing. Wound healing is directly associated with the migration speed of keratinocytes. The migration speed of human keratinocytes in presence of E1AM2, V8L (SEQ ID NO: 19), and Q19L variants is tested in comparison with pexiganan (SEQ ID NO: 54). Migration speed of human keratinocytes assessed in a 2D scratch assay at D 0 and D 2 timepoints. Decrease in wound area over time was measured by applying an ImageJ plugin ‘Wound Healing Size Tool’ on bright-field images. The change in wound area over the course of 48 hours was used as a measure of the migration speed of keratinocytes. Each value is the mean±SD (n=3). The p-value was calculated using ANOVA for multiple comparisons (Dunnett's multiple comparisons test). **** p<0.0001. *** p<0.001. ** p <0.01. * p<0.05. As can be seen from FIG. 7, both E1AM2 (3.125 μM-12.5 μM) and Q19L (3.125 μM-6.25 μM) increased the migration speed of keratinocytes significantly, implying wound healing ability. Both peptides healed wound faster within their respective MIC and MBC concentrations. V8L maintained keratinocyte migration speed, whereas Q19L showed significant migration delay only at the highest concentrations (100 μM). These results suggest that the peptide variants possess wound-healing ability apart from anti-bacterial efficacy. SEQ ID NO: 27=Q19L; SEQ ID NO: 2=E1AM2; SEQ ID NO: 19=V8L.

FIG. 8 shows that variant peptides such as E1AM2 (SEQ ID NO: 2), V8LQ19L (SEQ ID NO: 39), V8LQ19L C-2 (SEQ ID NO: 44) do not cause hemolysis in human red blood cells (hRBCs), suggesting minimal adverse effects in vivo. The hemolytic score of the peptide variants and pexiganan (SEQ ID NO: 54) are compared to a strong hemolytic peptide, melittin 12 as a control. As clearly shown in FIG. 8, none of the peptide variants or pexiganan caused significant hemolysis in hRBCs, even at high concentrations of 100 μM. E1AM2, V8LQ19L and V8LQ19L C-2 variants at their respective MIC/MBC concentrations showed ~0% hemolysis. The minimum peptide concentration corresponding to 10% hemolysis (minimal hemolysis concentration, HC) of E1AM2=50 μM, V8LQ19L=12.5 μM, V8LQ19L C-2=3.125 μM, and pexiganan=12.5 μM respectively. Readings of the melittin at 3.125 μM were normalised to 100%. Therefore, the variant peptides described herein do not exhibit cytotoxicity and hemolytic activities, which makes them suitable for in vivo applications.

FIG. 9 shows the synergistic effects of exemplary peptide variants in combination with antibiotics. FIG. 9A demonstrates reduced minimum inhibitory concentration (MIC) for E1AM2 D-V8L Q19L C-2-NH2 (SEQ ID NO: 46) against Pseudomonas aeruginosa (PA) in combination with exemplary antibiotic colistin. FIG. 9B demonstrates reduced minimum bactericidal concentration (MBC) for E1AM2 D-V8L Q19L C-2-NH2 (SEQ ID NO: 46) against Pseudomonas aeruginosa (PA) in combination with exemplary antibiotic colistin. FIG. 9C demonstrates reduced minimum inhibitory concentration (MIC) for E1AM2 W72Nal V8L Q19L C-2-NH2 (SEQ ID NO: 47) against Pseudomonas aeruginosa (PA) in combination with exemplary antibiotic colistin. FIG. 9D demonstrates reduced minimum bactericidal concentration (MBC) for E1AM2 W72Nal V8L Q19L C-2-NH2 (SEQ ID NO: 47) against Pseudomonas aeruginosa (PA) in combination with exemplary antibiotic colistin. FIG. 9E demonstrates reduced minimum inhibitory concentration (MIC) for E1AM2 D-W72Nal V8L Q19L C-2-NH2 (SEQ ID NO: 48) against Pseudomonas aeruginosa (PA) in combination with exemplary antibiotics gentamicin and piperacillin. FIG. 9F demonstrates reduced minimum bactericidal concentration (MBC) for E1AM2 D-W72Nal V8L Q19L C-2-NH2 (SEQ ID NO: 48) against Pseudomonas aeruginosa (PA) in combination with exemplary antibiotics gentamicin and piperacillin. In view of the results above, the peptide variants as described herein show synergistic anti-bacterial effects when used in combination with antibiotics.

FIG. 10 shows the potent fast-acting, broad-spectrum antibacterial effect of peptide V8LQ19L (SEQ ID NO: 39) against critical and high priority pathogens according to the World Health Organization (WHO), while also showcasing the lowest minimum inhibitory concentration (MIC) values. Peptide variant V8LQ19L offers a potent fast-acting, broad-spectrum antibacterial effect against critical and high priority pathogens World Health Organization (WHO), while showcasing the lowest MIC values. FIG. 10 also demonstrates MBC values (μM) for the peptide V8LQ19L, compared to pexiganan (SEQ ID NO: 54) and antibiotics against a range of broad-spectrum pathogens classified as critical and high priority by the WHO.

FIG. 11 demonstrates effective elimination of pathogens by peptide V8LQ19L (SEQ ID NO: 39) within the infected tissue of diabetic foot ulcer (DFU) patient. FIG. 11A shows the MBC values in μM for peptide V8LQ19L (SEQ ID NO: 39) and its D-variant (SEQ ID NO: 38) (unnatural amino acids) in comparison with pexiganan (SEQ ID NO: 54) and conventional clinical antiseptics, tested against pathogens isolated from the infected tissues of DFU patients. FIG. 11B shows images of the slurry resulting from bacterial enumeration post-sonication, alongside the bacteria isolated on plates. The varied colours and morphologies highlight the presence of multiple species and a diverse bacterial population within the infected tissues. Method: Freshly debrided infected tissues from DFU patients are weighed and immersed in 2 ml of MHB (Mueller-Hinton Broth) media for 48 hours to soften the tissues, facilitating the release of bacteria. These samples are subsequently sonicated for 1 minute at a 15A amplitude using a water bath sonicator to release the bacteria. The resultant slurry is plated on MHB media supplemented with 1.5% agar to isolate all bacteria. These bacterial isolates are then evaluated for their antibacterial efficacy following the CLSI (Clinical and Laboratory Standards Institute) guidelines.

FIG. 12 demonstrates killing of bacteria by the broad-spectrum antibacterial peptide E1AM2 V8LQ19L (SEQ ID NO: 39) primarily through membrane damage. FIG. 12A demonstrates live/dead visualization of PA performed using LIVE/DEAD BacLight Bacterial Viability and Counting Kit and visualized using an Olympus IX83 confocal microscope. Scale bar=3 μm. FIG. 12B demonstrates the comparison of the effects of E1AM2 V8LQ19L peptide with the effects of colistin, a membrane-disrupting antibiotic. The percentage of PA cells with (PI-positive, red) and without (Syto-9 stained, green) membrane damage following E1AM2 V8LQ19L peptide treatment are shown.

FIG. 13 demonstrates peptide V8LQ19L (SEQ ID NO: 39) even at its half-strength MBC level (0.78 μM) exhibiting a markedly reduced bacterial count with no observable remnants of biofilm material. V8LQ19L even at its half-strength MBC level (0.78 μM) exhibit a markedly reduced bacterial count with no observable remnants of biofilm material.

FIG. 14 demonstrates low toxicity and a broad therapeutic window of V8LQ19L (SEQ ID NO: 39) peptide variant in vitro. FIG. 14A demonstrates cell proliferation assays conducted on human keratinocytes (HaCaT) after 24 hours exposure to various doses of the peptide variant V8LQ19L and pexiganan (SEQ ID NO: 54). The peptide variant V8LQ19L was found to be noncytotoxic to HaCaT cells, even after 24 hours exposure at the highest tested concentrations. FIG. 14B demonstrates cell proliferation assays conducted on fibroblasts (HFF-1) after 24 hours exposure to various doses of the peptide variant V8LQ19L and pexiganan. The peptide variant V8LQ19L was found to be noncytotoxic to HFF-1 cells, even after 24 hours exposure at the highest tested concentrations. Intriguingly, HFF-1 cells treated with certain concentrations of both pexiganan and V8LQ19L were noted to exhibit enhanced proliferation. FIGS. 14A and 14B show that hydrogen peroxide (H₂O₂, 3%) that is commonly applied to chronic wounds as an antiseptic caused significant cytotoxicity to both HaCaT and HFF-1 cells, highlighting the risk some commercial antiseptic formulations might pose for dermal applications. Minimal toxicity observed for the V8LQ19L variant indicating that it is a safe treatment option. FIGS. 14C and 14D demonstrate the safety of peptide V8LQ19L via reduction of wound area over time under increased concentrations of peptide V8LQ19L on human keratinocytes. E1AM2 (V8LQ19L) did not impair wound healing even after 16X MBC levels corroborating findings of proliferation assays. FIG. 14C demonstrates that wound area reduction due to the lateral migration of human keratinocytes using a 2D scratch assay was analyzed at 2-h intervals, with increasing MBC of the V8LQ19L peptide. Wound closure was compared with the untreated samples (circle, red highlight). For improved clarity, error bars have been omitted from this representation. FIG. 14D shows the time required (hours) for complete wound closure plotted to emphasize early wound closure dynamics. Each value is the mean±SD (n=3). Statistical significance was assessed using ANOVA for multiple comparisons (Dunnett's multiple comparisons test). **** p<0.0001, *** p<0.001, ** p<0.01, *p<0.05, ns denotes nonsignificance, p >0.05.

FIG. 15 shows the evaluation of peptide V8LQ19L (SEQ ID NO: 39) gel for toxicity analysis. FIG. 15A shows macroscopic images demonstrating response to 1X-4X concentrations of V8LQ19L gel versus control gel, captured from day 0 to day 3. Scale: 1 cm. FIG. 15B shows quantitative analyses displaying the percentage change in wound area on day 1 for both V8LQ19L and control gel, relative to untreated wounds. Scale: 1 cm. FIG. 15C shows quantitative analyses displaying the percentage change in wound area on day 2 for both V8LQ19L and control gel, relative to untreated wounds. Scale: 1 cm. FIG. 15D shows quantitative analyses displaying the percentage change in wound area on day 3 for both V8LQ19L and control gel, relative to untreated wounds. Scale: 1 cm. FIG. 15E shows quantitative analyses displaying overall time progression for both V8LQ19L and control gel, relative to untreated wounds. Scale: 1 cm. FIG. 15F shows H&E-stained images of the wound treated with V8LQ19L gel, highlighting nascent epidermis (marked with yellow circles). FIG. 15G shows the analysis of average re-epithelialization percentage post-treatment with V8LQ19L gel. Scale bar: 500 μm. FIG. 15H shows representative H&E images of wound tissues from varying concentrations of V8LQ19L hydrogel treatments at day 3, focusing on the inflammation region. Amplified inflammation region scale=50 μm, biological replicates, n=6-12.

FIG. 16 demonstrates significant bacterial clearance at the wound site using V8LQ19L (SEQ ID NO: 39) gel at 1X MBC compared with treatment with hydrogel alone. Excisional wounds infected with PA for 24 hours being treated for 24 hours with the E1AM2 V8LQ19L hydrogel at its 1X MBC concentration, compared with hydrogel-only treatment in a mouse model. V8LQ19L gel at 1X MBC reduced PA infection based on viable CFU counts. Each circle in the graph represents one mouse. Statistical significance was determined using a t-test, with *p<0.05. The data are presented as colony-forming unit counts presented as means±SEM. In a PA-induced mouse excisional wound infection model, V8LQ19L gel at 1X MBC reduced PA infection based on viable CFU counts, indicating significant bacterial clearance at the wound site compared with treatment with hydrogel alone. Collectively, the E1AM2 V8LQ19L peptide showcases unique functionality, combining antibacterial effectiveness and biocompatibility for dermal applications.

### Definitions

As used herein, the term “unnatural amino acids” refers to non-coded or non-proteinogenic amino acids which are distinct from the 22 proteinogenic amino acids (21 in eukaryotes) which are naturally encoded in the genome of organisms for the assembly of proteins. Unnatural amino acids can occur naturally or be synthesized in the laboratory. For example, the amino acid is a D-amino acid. As exemplified in the present disclosure, the unnatural amino acids can be, but are not limited to:

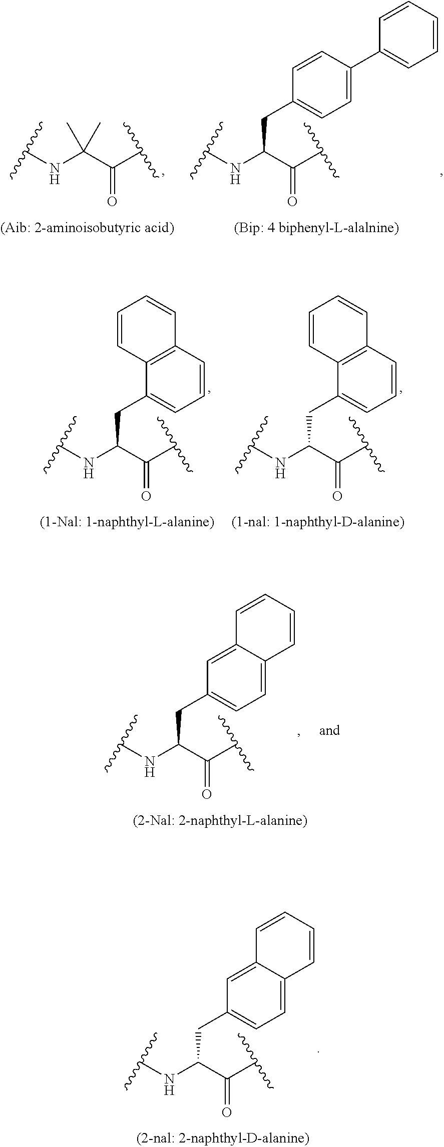

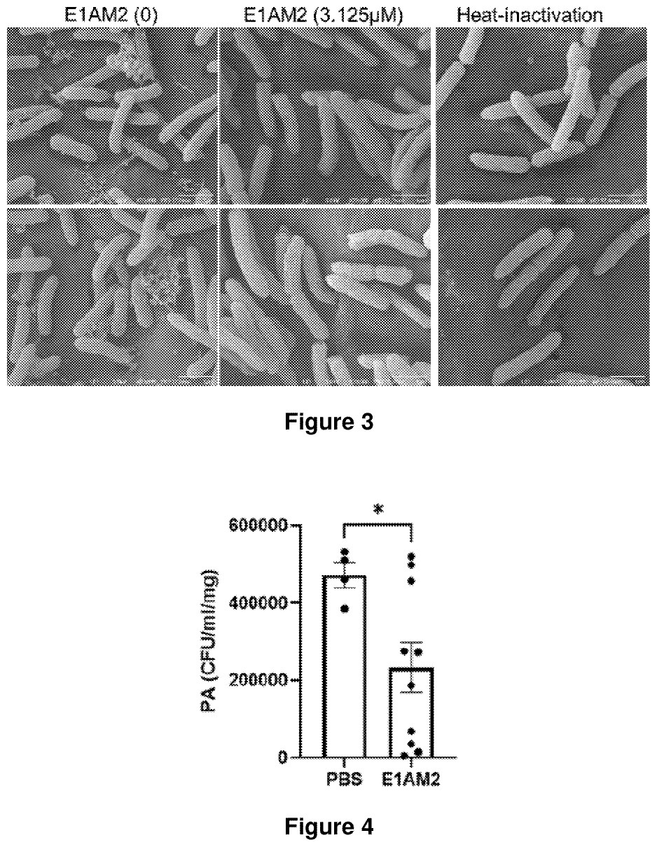

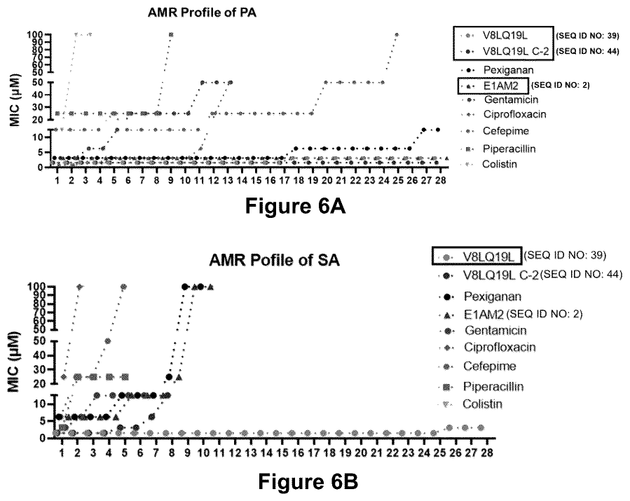

As used herein, the term “biofilm” is defined as a syntrophic community of microorganisms attached to an inert or living surface by a self-produced polymeric matrix or an assemblage of microbial cells associated with a surface and enclosed in a matrix of primarily polysaccharide material. Biofilms have been found to be involved in a wide variety of microbial infections in the body, and bacterial biofilms may impair cutaneous wound healing and reduce topical anti-bacterial efficiency in healing or treating infected skin wounds. For example, biofilm formation of Pseudomonas aeruginosa, along with other bacteria, is found in 90% of chronic wound infections, which leads to poor healing and high cost of treatment. Streptococcus pneumoniae is the main cause of community-acquired pneumonia and meningitis in children and the elderly, and of sepsis in HIV-infected persons. Escherichia coli biofilms are responsible for many intestinal infectious diseases. Staphylococcus aureus infects skin and lungs, leading to skin infection and pneumonia. The biofilm infections network of Staphylococcus aureus plays a critical role in preventing immune cells, such as macrophages from eliminating and destroying bacterial cells. As clearly shown in the SEM images in FIG. 3, peptide variants disclosed herein, such as E1AM2 disintegrate biofilm effectively. Furthermore, biofilm formation by bacteria, such as Staphylococcus aureus, not only develop resistance against antibiotic medication but also develop internal resistance toward antimicrobial peptides (AMPs), leading to preventing the inhibition of the pathogen and maintaining its survival. Contrary to the conventional AMPs, as demonstrated in the present application in FIG. 6, for example, the variant peptides disclosed herein maintain effective bactericidal ability against the exemplary bacterial strains even after long-term treatment.

As used herein, the term “chronic wounds” refers to a wound that cannot achieve anatomical and functional integrities through normal, orderly, and timely repair processes under the influence of various internal or external factors. Wound healing, as a normal biological process in the human body, is achieved through four precisely and highly programmed phases: hemostasis, inflammation, proliferation, and remodeling. For a wound to heal successfully, all four phases must occur in the proper sequence and time frame. Chronic wounds are injuries that have not healed and have no tendency to heal after more than an extended period, for example, one month of treatment. Examples of chronic wounds include, nonhealing or infected surgical or traumatic wounds, venous ulcers, pressure ulcers, diabetic foot ulcers, and ischemic ulcers.

As used herein, the term “nosocomial infection” or “healthcare-associated infections (HAI)” refers to an infection that is not present or incubating at the time of admission to a treatment and occurs during or after treatment. These infections include catheter-associated urinary tract infections, central line-associated bloodstream infections, surgical site infections, ventilator-associated pneumonia, hospital-acquired pneumonia, and Clostridium difficile infections.

As used herein, the term “diabetes mellitus”, or “diabetes” refers to a heterogeneous complex metabolic disorder characterized by hyperglycemia, i.e. elevated blood glucose concentration secondary to either resistance to the action of insulin, insufficient insulin secretion, or both. Commonly, diabetes can be classified into two categories, the type 1 and type 2 diabetes. Type 1 diabetes is characterized by an absolute deficiency of insulin secretion. Individuals at increased risk of developing this type of diabetes can often be identified by serological evidence of an autoimmune pathologic process occurring in the pancreatic islets and by genetic markers. For type 2 diabetes, the cause is a combination of resistance to insulin action and an inadequate compensatory insulin secretory response. Statistically, most patients with type 2 diabetes are obese, and obesity itself causes some degree of insulin resistance. Diabetic patients are in particular susceptible to infections, which accompany chronic hyperglycemia.

As used herein, the term “diabetic foot ulcer (DFU)” or “diabetic foot infections (DFI)” is one of the most common and challenging complication associated with the onset of diabetes mellitus, which lowers the quality of life and results in significant morbidity and mortality for patients. In diabetic patients, the chronic foot ulcer is typically caused by poor blood circulation and peripheral neuropathy, which is localized nerve damage. Treatment for diabetic foot ulcer (DFU) is particularly challenging due to the high recurrence rate and antibiotic resistance.

As used herein, the term “overweight” refers to a condition of excessive fat deposits. As used herein, the term “obesity” refers to a chronic complex disease defined by excessive fat deposits that can impair health, according to World health Organization (WHO). Obesity can lead to increased risk of type 2 diabetes and heart disease, it can affect bone health and reproduction, it increases the risk of certain cancers. Obesity influences the quality of living, such as sleeping or moving. Typically for adults, WHO defines overweight as having a BMI greater than or equal to 25 and obesity as having a BMI greater than or equal to 30.

As used herein, the term “microbe” or “microorganism” refers broadly to bacteria, protozoa, algae, and fungi.

As used herein “antimicrobial resistance (AMR)” or “drug resistance” refers to the ability for pathogens to evade the killing of anti-microbials. Pathogens with antimicrobial resistance (AMR) no longer responds to medicines, thus making infections harder to treat and increasing the risk of disease spread, severe illness and death. Anti-microbial agents include, and are not limited to antibiotics, anti-virals, anti-fungals and anti-parasitics. Anti-microbial agents are medicines used to prevent and treat infections in humans, animals and plants.

As used herein, the term “multidrug resistant (MDR) pathogens” refers to pathogens that have become resistant to certain antibiotics, and these antibiotics can no longer be used to control or kill the pathogen. Such pathogens include, but are not limited to fungi, protists, and bacteria. Exemplary multidrug-resistant fungi include Candida auris, which is resistant to multiple antifungal drugs commonly used to treat Candida infections. Some strains are resistant to all three available classes of antifungals drugs. Multidrug resistant bacteria are commonly found in healthcare facilities, elderly care centers, for examples, and include bacteria strains such as methicillin-resistant Staphylococcus aureus (MRSA), vancomycin-resistant Enterococci (VRE), extended-spectrum β-lactamase (ESBLs) producing Gram-negative bacteria, and Klebsiella pneumoniae carbapenemase (KPC) producing Gram-negative bacteria.

As used herein, the term “ESKAPE pathogen” is an acronym comprising the scientific names of six highly virulent and antibiotic resistant bacterial pathogens, which are Enterococcus faecium, Staphylococcus aureus, Klebsiella pneumoniae, Acinetobacter baumannii, Pseudomonas aeruginosa, and Enterobacter spp. These bacterial pathogens can evade or ‘escape’ commonly used antibiotics due to their increasing multi-drug resistance (MDR).

As used herein, the term “anti-bacterial” or “bactericidal” are used interchangeably and are used to refer to the ability or characteristics of killing of bacteria cells and/or controlling the development of bacterial infection in a tissue, an organ, or a subject.

As used herein, the term “angiogenesis” refers to the growth of blood vessels. The term “angiogenesis-inducing growth factors” refers to growth factors that induce the growth of blood vessels, such as vascular endothelial growth factor (VEGF) or basic fibroblast growth factor (bFGF).

As used herein, the term “biofilm” refers to a membranous tissue formed by pathogens such as bacteria attached to a chronic wound bed and fused with extracellular matrix secreted by the film. It is composed of pathogens and their products, extracellular matrix, necrotic tissue, and so on. Clinically, biofilm is more common in pressure ulcer, diabetic foot ulcer, lower extremity arteriovenous ulcer, and other chronic wounds.

As used herein, the term “minimum inhibitory concentration (MIC)” measures the in vitro levels of susceptibility or resistance of specific bacterial strains to applied antibiotic. Minimum inhibitory concentration (MIC) is the lowest concentration of an anti-bacterial agent expressed in mg/L (μg/mL) which, under strictly controlled in vitro conditions, completely prevents >99% of visible growth of the test strain of an organism.

As used herein, the term “minimum bactericidal concentration (MBC)” measures the lowest concentration of anti-bacterial agent required to kill >99% of a particular bacterium.

As used herein, the term “minimum biofilm inhibitory concentration (MBIC)” is defined as the lowest concentration of the anti-bacterial agent to inhibit the initial formation of biofilm. At this concentration, there is no time-dependent increase in the average number of viable cells in the biofilm.

As used herein, the term “colony forming unit (CFU)” refers to a unit which estimates the number of microbial cells in a sample that are viable, able to multiply via binary fission under the controlled conditions. The higher the CFU is, the more microbial cells there are in the given sample.

### DETAILED DESCRIPTION

Chronic wound management is a large and growing problem in healthcare. As of today, antibiotics stand as the first line of treatment against wound infections. However, due to the drug resistance in most chronic wound causing pathogens, standard antibiotics treatments show little effect. The emergence of antibiotic resistant infections accompanied with the dearth of new antibiotics poses the biggest challenge. What is needed is an anti-bacterial agent effective in treating chronic infections, without developing significant antimicrobial resistance (AMR).

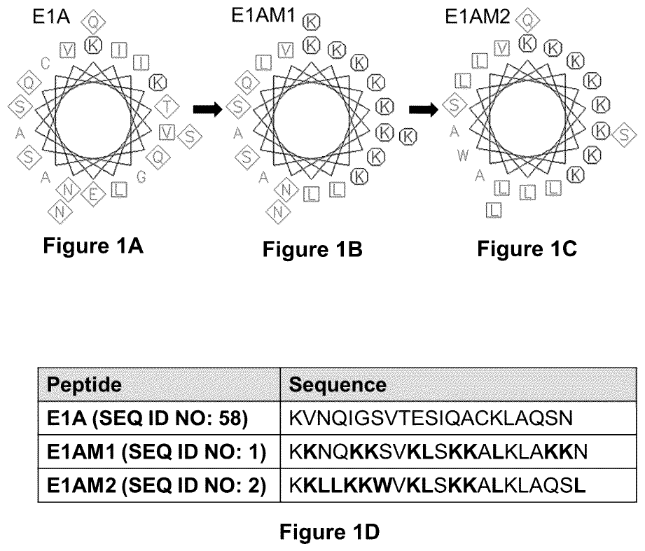

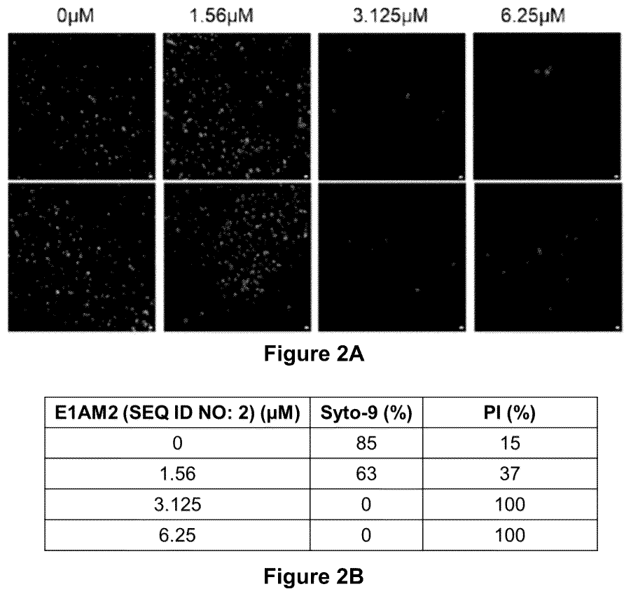

Therefore, the present disclosure provides anti-bacterial peptides that can be used to effectively treat chronic infections, in particular those caused by anti-microbial resistant pathogens. The anti-bacterial peptides described herein are variants of the base sequence of EIA (KVNQIGSVTESIQACKLAQSN). As used herein, the term “variant peptides” or “peptide variants” both refer to the peptides as disclosed herein based on E1A. E1A is a 21-residue short region within peptide ENO3 (SEQ ID NO: 52). The variant contains one or more additional modifications compared to the base sequence of E1A, which include but are not limited to, deletion, insertion, point mutation, amidation, acetylation, or a combination thereof. For example, as demonstrated in FIG. 1A and FIG. 1B in comparison, the positively charged, basic lysine residues are separated from other non-polar or polar (acid or uncharged) residues and negatively charged glutamate (10) are substituted to leucine to prevent possible repulsion of the peptide in contact with bacterial membrane. The peptide variants described herein contain cationic arms which disrupt the bacterial membrane to achieve the bactericidal effects, as demonstrated in the staining results of FIG. 2, for example.

In one aspect, the peptides described herein comprise a general sequence of KKLLKKX₁X₂KLX₃KKX₄L (SEQ ID NO: 49). Within said general sequence, variable amino acids are defined such that X₁ can be any one of: S, W, L, K, 4-biphenyl-L-alalnine, 1-naphthyl-L-alanine, and 2-naphthyl-L-alanine. Within said general sequence, variable amino acids are defined such that X₂ can be any one of: V, L, and K. Within said general sequence, variable amino acids are defined such that X₃ can be any one of: S, L, and K. Within said general sequence, variable amino acids are defined such that X₄ can be any one of: A, L, K, and 2-aminoisobutyric acid. Therefore, the anti-bacterial peptide described herein can be defined as comprising the sequence of KKLLKKX₁X₂KLX₃KKX₄L (SEQ ID NO: 49), with X₁ being a S, W, L, K, 4-biphenyl-L-alalnine, 1-naphthyl-L-alanine, or 2-naphthyl-L-alanine; X₂ being a V, L, or K; X₃ being a S, L, or K; and X₄ being an A, L, K, or 2-aminoisobutyric acid.

In another aspect, the peptides as described herein comprise the general sequence of X₁X₂KLX₃KKX₄LKLX₅X₆X₇L (SEQ ID NO: 50). Within said general sequence, variable amino acids are defined such that X₁ can be any one of: S, W, L, K, 4-biphenyl-L-alalnine, 1-naphthyl-L-alanine, or 2-naphthyl-L-alanine. Within said general sequence, variable amino acids are defined such that X₂ can be any one of: V, L, and K. Within said general sequence, variable amino acids are defined such that X₃ can be any one of: S, L, and K. Within said general sequence, variable amino acids are defined such that X₄ can be any one of: A, L, K, and 2-aminoisobutyric acid. Within said general sequence, variable amino acids are defined such that X₅ can be any one of: A, L, K, and 2-Aminoisobutyric acid. Within said general sequence, variable amino acids are defined such that X₆ can be any one of: Q, L, and K. Within said general sequence, variable amino acids are defined such that X₇ can be any one of: S, L, and K. Therefore, the anti-bacterial peptide described herein can be defined as comprising the sequence of X₁X₂KLX₃KKX₄LKLX₅X₆X₇L (SEQ ID NO: 50), with X₁ being a S, W, L, K, 4-biphenyl-L-alalnine, 1-naphthyl-L-alanine, or 2-naphthyl-L-alanine; X₂ being V, L, or K; X₃ being a S, L, or K; X₄ being an A, L, K, or 2-aminoisobutyric acid; X₅ being an A, L, K, or 2-aminoisobutyric acid; X₆ being a Q, L, or K; and X₇ being a S, L, or K. In one example, the peptide variant is E1AM1 having a sequence of KKNQKKSVKLSKKALKLAKKN (SEQ ID NO: 1). In another example, the peptide variant is E1AM2 having a sequence of KKLLKKWVKLSKKALKLAQSL (SEQ ID NO: 2).

The peptide variants as described herein, for example, E1AM1 (SEQ ID NO: 1) and E1AM2 (SEQ ID NO: 2) exhibit anti-bacterial effects against wound infecting pathogens. To evaluate their anti-bacterial potency, broth microdilution method as per the CLSI guidelines 1 was used. Antibiotics commonly used against chronic wound infections such as colistin, gentamicin, piperacillin, ciprofloxacin, and cefepime are used as positive control. Pexiganan, a potent antimicrobial peptide of the art (topical antimicrobial therapy for diabetic foot ulcer infections in Phase III clinical trial (NCT01590758)). The anti-bacterial potency of the tested agents is quantified using minimum inhibitory concentration (MIC) and minimum bactericidal concentration (MBC). The MIC is defined as the lowest compound concentration (μM) which can inhibit bacterial growth after an overnight incubation to >99% based on the absorbance readings at 600 nM. To obtain the MBC data, 1X, 2X and 4× MIC of the peptide variants were plated to obtain the number of viable colonies compared to the original inoculum. Lowest compound concentration (μM) showing >99% bacterial killing is indicated as their MBC values. As shown in Table 1 below, E1AM2 displayed lower MIC of 3.125 μM and MBC of 6.25 μM against Pseudomonas aeruginosa (PA), comparable to Pexiganan. E1AM2 displayed a much lower MIC of 6.25 μM and MBC of 12.5 μM against Staphylococcus aureus (SA), wherein pexiganan showed a two-higher value, indicating lower anti-bacterial efficacy. Overall, against both the bacterial strains evaluated, the exemplary variant peptides demonstrated improved or comparable level of anti-bacterial potency compared to known antibiotics and peptides.

TABLE 1
MICs (μM) and MBC (μM) of peptides against clinical
isolates of Pseudomonas aeruginosa (PA) and
Staphylococcus aureus (SA)

| | MIC | MBC |
|---|---|---|
| | (μM) | (μM) |
| Pseudomonas aeruginosa (PA) | | |
| Pexiganan | 3.125 | 6.25 |
| E1AM2-NH2 (SEQ ID NO: 2) | 3.125 | 6.25 |
| Colistin | 3.125 | 6.25 |
| Gentamicin | 3.125 | 6.25 |
| Piperacillin | 25 | 50 |
| Ciprofloxacin | 0.78 | 1.56 |
| Cefepime | 12.5 | 25 |
| Staphylococcus aureus (SA) | | |
| Pexiganan | 6.25 | 12.5 |
| E1AM2-NH2 (SEQ ID NO: 2) | 6.25 | 12.5 |
| Colistin | >100 | >200 |
| Gentamicin | 1.56 | 3.25 |
| Piperacillin | 6.25 | 12.5 |
| Ciprofloxacin | 25 | 50 |
| Cefepime | 12.5 | 25 |

The peptide variants as disclosed herein can comprise additional amino acids further to the sequences defined above. For example, the peptide variants comprise additional amino acids at the C-terminus. As exemplified in Table 2 of the present application, the peptide variant E1AM2 C-1-NH2 has a sequence of KKLLKKWVKLSKKALKLAQS (SEQ ID NO: 11); the peptide variant E1AM2 C-2-NH2 has a sequence of KKLLKKWVKLSKKALKLAQ (SEQ ID NO: 12); the peptide variant E1AM2 C-3-NH2 has a sequence of KKLLKKWVKLSKKALKLA (SEQ ID NO: 13); the peptide variant E1AM2 C-4-NH2 has a sequence of KKLLKKWVKLSKKALKL (SEQ ID NO: 14); the peptide variant E1AM2 C-5-NH2 has a sequence of KKLLKKWVKLSKKALK (SEQ ID NO: 15); the peptide variant E1AM2 C-6-NH2 has a sequence of KKLLKKWVKLSKKAL (SEQ ID NO: 16).

In a further example, the peptide variants comprise additional amino acids at the N-terminus. As exemplified in Table 2 of the present application, the peptide variant E1AM2 N-1-NH2 has a sequence of KLLKKWVKLSKKALKLAQSL (SEQ ID NO: 5); the peptide variant E1AM2 N-2-NH2 has a sequence of LLKKWVKLSKKALKLAQSL (SEQ ID NO: 6); the peptide variant E1AM2 N-3-NH2 has a sequence of LKKWVKLSKKALKLAQSL (SEQ ID NO: 7); the peptide variant E1AM2 N-4-NH2 has a sequence of KKWVKLSKKALKLAQSL (SEQ ID NO: 8); the peptide variant E1AM2 N-5-NH2 has a sequence of KWVKLSKKALKLAQSL (SEQ ID NO: 9); the peptide variant E1AM2 N-6-NH2 has a sequence of WVKLSKKALKLAQSL (SEQ ID NO: 10). In some examples, the anti-bacterial peptide as described herein has less than 22 amino acid residues.

The minimum inhibitory concentration (MIC) values for the peptide variants comprising additional amino acids further to the general sequences defined are shown in Table 2 below, for example. While the MIC values vary across different peptide variants, the overall values are within the range for effective killing of bacteria.

TABLE 2
MICs (μM) of E1AM2 peptide variants against
Pseudomonas aeruginosa (PA)

| SEQ | | | |
|---|---|---|---|
| ID | Peptides | | MIC PA |
| NOS | (C-amidated) | Sequence | (μM) |
| 2 | E1AM2 | KKLLKKWVKLSKKALKLAQSL | 3.125 |
| 5 | E1AM2 N-1 | KLLKKWVKLSKKALKLAQSL | 12.5 |
| 6 | E1AM2 N-2 | LLKKWVKLSKKALKLAQSL | 25 |
| 7 | E1AM2 N-3 | LKKWVKLSKKALKLAQSL | 12.5 |
| 8 | E1AM2 N-4 | KKWVKLSKKALKLAQSL | >25 |
| 9 | E1AM2 N-5 | KWVKLSKKALKLAQSL | >25 |
| 10 | E1AM2 N-6 | WVKLSKKALKLAQSL | >25 |
| 11 | E1AM2 C-1 | KKLLKKWVKLSKKALKLAQS | >25 |
| 12 | E1AM2 C-2 | KKLLKKWVKLSKKALKLAQ | 25 |
| 13 | E1AM2 C-3 | KKLLKKWVKLSKKALKLA | 6.25 |
| 14 | E1AM2 C-4 | KKLLKKWVKLSKKALKL | >12.5 |
| 15 | E1AM2 C-5 | KKLLKKWVKLSKKALK | >25 |
| 16 | E1AM2 C-6 | KKLLKKWVKLSKKAL | >25 |

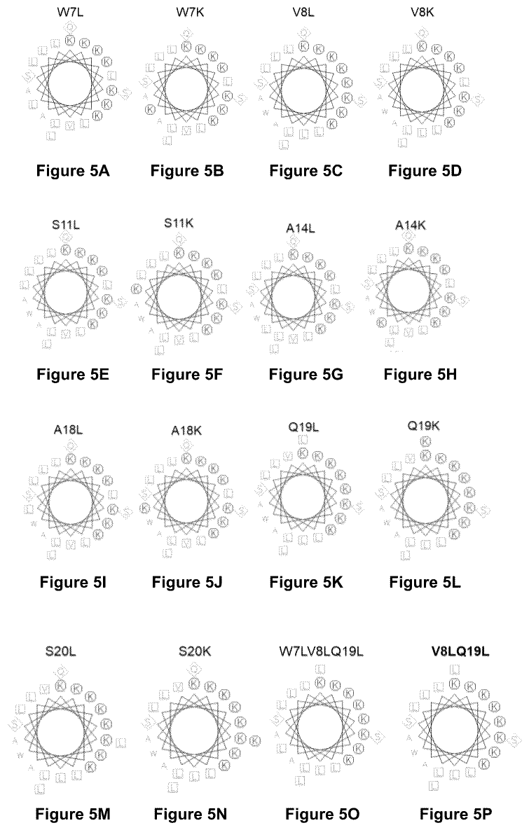

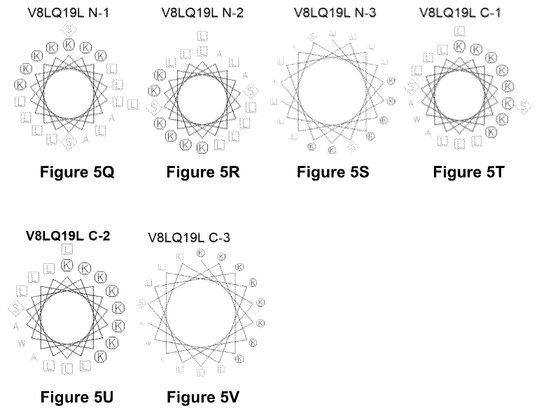

The variant peptides as disclosed herein can comprise substitution of one or more amino acid residues compared to the E1A base sequence. For example, the substitution can be from an amino acid residue to leucine (L) and/or lysine (K) residues to increase anti-bacterial potency and reduce hemolytic activity, while keeping the cationic arm intact. As exemplified in FIG. 5, and Table 3 below, variants Q19L and V8L (bolded and underlined) exhibited reduction in the minimum inhibitory concentration (MIC) and minimum bactericidal concentration (MBC) values compared to E1AM2 and pexiganan against both Pseudomonas aeruginosa (PA) and Staphylococcus aureus (SA). Variant W7L also exhibited reduced MIC against PA.

TABLE 3
MICs (μM) and MBC (μM) of E1AM2 variants against Pseudomonas aeruginosa (PA)
and Staphylococcus aureus (SA)

| | | | PA | | SA | |
|---|---|---|---|---|---|---|
| | Peptides | | MIC | MBC | MIC | MBC |
| SEQ ID NOS | (C-amidated) | Sequence | (μM) | (μM) | (μM) | (μM) |
| 54 | PEX | GIGKF LKKAK KFGKA FVKIL KK | 3.128 | 6.25 | 6.25 | 12.8 |
| 2 | E1AM2 | KKLLK KWVKLSKKALKLAQSL | 3.126 | 6.25 | 6.25 | 12.5 |
| 17 | W7L | KKLLK KLVKL SKKAL KLAQS L | 3.125 | 3.125 | 6.25 | 6.25 |
| 18 | W7K | KKLLK KKVKL SKKAL KLAQS L | 12.5 | 12.5 | 12.5 | 25 |
| 19 | V8L | KKLLK KWLKL SKKAL KLAQS L | 3.125 | 3.125 | 3.125 | 3.125 |
| 20 | V8K | KKLLK KWKKL SKKAL KLAQS L | 3.125 | 3.126 | 12.5 | 12.5 |
| 21 | S11L | KKLLK KWVKL LKKAL KLAQS L | 12.5 | 12.5 | 6.25 | 6.25 |
| 22 | S11K | KKLLK KWVKL KKKAL KLAQS L | 12.5 | 12.5 | 12.5 | 12.5 |
| 23 | A14L | KKLLK KWVKL SKKLL KLAQS L | 12.5 | 12.5 | 6.25 | 6.25 |
| 24 | A14K | KKLLK KWVKL SKKKL KLAQS L | 12.5 | 12.5 | 12.5 | 12.5 |
| 25 | A18L | KKLLK KWVKL SKKAL KLLQS L | 6.25 | 6.25 | 3.125 | 3.125 |
| 26 | A18K | KKLLK KWVKL SKKAL KLKQS L | 12.5 | 25 | 12.5 | 12.5 |
| 27 | Q19L | KKLLK KWVKL SKKAL KLALS L | 3.125 | 3.125 | 3.125 | 3.125 |
| 28 | Q19K | KKLLK KWVKL SKKAL KLAKS L | 12.5 | 25 | 12.5 | 25 |
| 29 | S20L | KKLLK KWVKL SKKAL KLAQL L | 6.25 | 12.5 | 6.25 | 12.5 |
| 30 | S20K | KKLLK KWVKL SKKAL KLAQK L | 25 | 25 | 25 | 25 |

Variant peptides with combinations of substitutions also exhibit bactericidal activity as demonstrated in Table 4 below. All the 14 exemplary peptides with multiple substitutions maintain the strong anti-bacterial activity as can be seen from the MIC and MBC values against exemplary bacterial strains Pseudomonas aeruginosa (PA) and Staphylococcus aureus (SA). Variant containing Q19L and V8L substitutions in combination shows synergistic effects by exhibiting reduced MIC/MBC of 1.56 μM against both PA and SA, followed by W7L substitution.

TABLE 4
MICs (μM) and MBC (μM) of V8LQ19LW7L and V8LQ19L variants against
both Pseudomonas aeruginosa (PA) and Staphylococcus aureus (SA)

| | | | PA | | SA | |
|---|---|---|---|---|---|---|
| | Peptides | | MIC | MBC | MIC | MBC |
| SEQ ID NOS | (C-amidated) | Sequence | (μM) | (μM) | (μM) | (μM) |
| 54 | PEX | GIGKF LKKAK KFGKA FVKIL KK | 3.125 | 8.25 | 6.25 | 12.5 |
| 2 | E1AM2 | KKLLK KWVKLSKKALKLAQSL | 3.125 | 6.25 | 6.25 | 12.5 |
| 39 | V8LQ19L | KKLLK KWLKL SKKAL KLALS L | 1.56 | 1.56 | 1.56 | 1.56 |
| 34 | W7LV8LQ19L | KKLLK KLLKL SKKAL KLALS L | 3.125 | 3.125 | 1.58 | 1.58 |

Therefore, in another aspect, the peptide as described herein comprises the general sequence of KKLLKKX₁X₂KLX₃KKX₄LKLX₅X₆X₇L (SEQ ID NO: 51). Within said general sequence, variable amino acids are defined such that X₁ can be any one of: S, W, L, K, 4-biphenyl-L-alalnine, 1-naphthyl-L-alanine, or 2-naphthyl-L-alanine. Within said general sequence, variable amino acids are defined such that X₂ can be any one of: V, L, and K. Within said general sequence, variable amino acids are defined such that X₃ can be any one of: S, L, and K. Within said general sequence, variable amino acids are defined such that X₄ can be any one of A, L, K, and 2-aminoisobutyric acid. Within said general sequence, variable amino acids are defined such that X₅ can be any one of A, L, K, and 2-aminoisobutyric acid. Within said general sequence, variable amino acids are defined such that X₆ can be any one of Q, L, and K. Within said general sequence, variable amino acids are defined such that X₇ can be any one of S, L, and K. Therefore, the anti-bacterial peptide described herein can be defined as comprising the sequence of KKLLKKX₁X₂KLX₃KKX₄LKLX₅X₆X₇L (SEQ ID NO: 51), with X₁ being a S, W, L, K, 4-biphenyl-L-alalnine, 1-naphthyl-L-alanine, or 2-naphthyl-L-alanine; X₂ being V, L, or K; X₃ being a S, L, or K; X₄ being an A, L, K, or 2-Aminoisobutyric acid; X₅ being an A, L, K, or 2-Aminoisobutyric acid; X₆ being a Q, L, or K; and X₇ being a S, L, or K.

The anti-bacterial peptide as described herein can comprise one or more unnatural amino acids, which improves the stability of the peptides. The unnatural amino acids as exemplified in the present application include, but are not limited to the following:

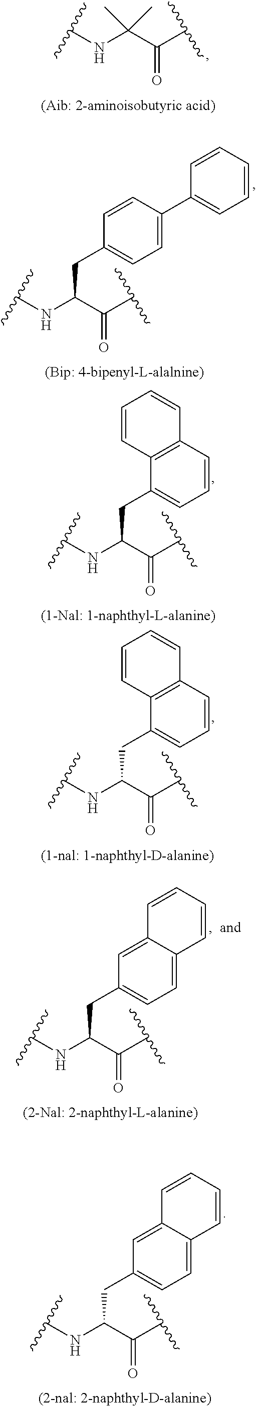

For example, helix-inducing unnatural amino acid aminoisobutyric acid (Aib) is used to replace alanine at positions 14 & 18 on exemplary peptide V8LQ19L. As shown in Table 5, such substitution does not compromise the anti-bacterial activity of the variant peptides. Aib substitution even increased the minimum inhibitory concentration (MIC) and minimum bactericidal concentration (MBC) values against Pseudomonas aeruginosa (PA) by 2 folds. A14Aib replacement increased the MIC/MBC values by 2 folds against Staphylococcus aureus (SA).

TABLE 5
MICs (μM) and MBC (μM) of V8LQ19L variant upon alanine substitution with Aib
against both Pseudomonas aeruginosa (PA) and Staphylococcus aureus (SA)

| | | | PA | | SA | |
|---|---|---|---|---|---|---|
| | Peptides | | MIC | MBC | MIC | MBC |
| SEQ ID NOS | (C-amidated) | Sequence | (μM) | (μM) | (μM) | (μM) |
| 54 | PEX | GIGKF LKKAK KFGKA FVKIL KK | 3.128 | 6.25 | 6.26 | 12.5 |
| 2 | E1AM2 | KKLLK KWVKLSKKALKLAQSL | 3.125 | 8.26 | 6.25 | 12.5 |
| 39 | VBLQ19L | KKLLK KWVLKL SKKAL KLALS L | 1.58 | 1.58 | 1.56 | 1.56 |
| 31 | VSLQ19LA14Aib | KKLLK KWLKL SKK(Aib)L KLALS L | 3.126 | 6.25 | 3.125 | 6.25 |
| 32 | V8LQ19LA18Aib | KKLLK KWLKL SKKAL KL(Aib)LS L | 3.125 | 3.125 | 1.58 | 1.56 |
| 33 | V8LQ19LA1418Aib | KKLLK KWLKL SKK(Aib)L KL(Aib)LS L | 3.126 | 3.125 | 1.56 | 1.56 |

In another example, the animo acid residue within the peptide variant can be replaced by unnatural amino acids such as 4-biphenyl-L-alalnine (Bip), 1-naphthyl-L-alanine (1-Nal), and 2-naphthyl-L-alanine (2-Nal). Bip substitution can improve bacterial membrane disruption, while 1-Nal and 2-Nal increase the stability of the peptide against proteases. As a demonstration, W7 position of the peptide is substituted with Bip, 1-Nal, or 2-Nal. Table 6 below shows the respective minimum inhibitory concentration (MIC) and minimum bactericidal concentration (MBC) values against Pseudomonas aeruginosa (PA) and Staphylococcus aureus (SA) at W7 position of the peptide. Against SA, W7 substitution showed comparable MIC/MBC levels to V8LQ19L variant. Bip substitution increased the MIC/MBC by 2 folds, while the other two remained unchanged.

In another example, the animo acid residue within the peptide variant can be replaced by unnatural amino acids such as D-form amino acids. Exemplary peptide D-V8LQ19L is shown in Table 6, which retains the strong anti-bacterial effects as previously demonstrated.

TABLE 6
MICs (μM) and MBC (μM) of W7 substitution of V8LQ19L variant against both
Pseudomonas aeruginosa (PA) and Staphylococcus aureus (SA)

| | | | PA | | SA | |
|---|---|---|---|---|---|---|
| | Peptides | | MIC | MBC | MIC | MBC |
| SEQ ID NOS | (C-amidated) | Sequence | (μM) | (μM) | (μM) | (μM) |
| 54 | PEX | GIGKF LKKAK KFGKA FVKIL KK | 3.125 | 6.25 | 6.25 | 12.6 |
| 2 | E1AM2 | KKLLK KWVKLSKKALKLAQSL | 3.125 | 8.25 | 6.25 | 12.5 |
| 39 | V8LQ19L | KKLLK KWLKL SKKAL KLALS L | 1.56 | 1.56 | 1.56 | 1.56 |
| 35 | W7BipV8LQ19L | KKLLK K(Bip)LKL SKKAL KLALS L | 3.325 | 3.125 | 1.58 | 1.56 |
| 36 | W71NalV8LQ19L | KKLLK K(1-Nal)LKL SKKAL KLALS L | 3.125 | 3.125 | 1.56 | 1.56 |
| 37 | W72NalV8LQ19L | KKLLK K(2-Nal)LKL SKKAL KLALS L | 1.56 | 1.66 | 1.56 | 1.56 |
| 38 | D-V8LQ19L | kklik kwlkl skkal klais i | 3.125 | 3.125 | 3.125 | 3.125 |

Apart from the exemplary substitutions provided above, other choices of unnatural amino acids are known in the art and a person skilled in the art would appreciate that such substitution does not compromise the anti-bacterial potency from the numerous examples provided herein.

In some examples, additional variants combining amino acid substitutions and the incorporation of additional amino acids further to the general sequences defined above shows comparable or improved efficiency in killing bacteria. According to Table 7 below, variants V8LQ19L N-1, V8LQ19L N-2, V8LQ19L N-3, V8LQ19L C-1, V8LQ19L C-2, and V8LQ19L C-3, which comprise both types of variations in their sequences, demonstrate strong potency against both Pseudomonas aeruginosa (PA) and Staphylococcus aureus (SA).

TABLE 7
MICs (μM) of V8LQ19L deletion variants against both Pseudomonas aeruginosa (PA)
and Staphylococcus aureus (SA)

| | | | PA | | SA | |
|---|---|---|---|---|---|---|
| | Peptides | | MIC | MBC | MIC | MBC |
| SEQ ID NOS | (C-amidated) | Sequence | (μM) | (μM) | (μM) | (μM) |
| 54 | PEX | GIGKF LKKAK KFCKA FVKIL KK | 3.125 | 6.25 | 6.25 | 12,5 |
| 2 | E1AM2 | KKLLK KWVKLSKKALKLAQSL | 3.128 | 6.25 | 6.28 | 12.5 |
| 39 | V8LQ19L | KKLLK KWLKL SKKAL KLALS L | 1.56 | 1.56 | 1.56 | 1.56 |
| 40 | VBLQ19L N-1 | LLK KWLKL SKKAL KLALS L | 3.125 | 3.125 | 1.56 | 1.56 |
| 41 | VBLQ19L N-2 | LK KWLKL SKKAL KLALS L | 6.25 | 6.25 | 3.125 | 3.12 |
| 42 | VSLQ19L N-3 | K KWLKL SKKAL KLALS L | 8.25 | 6.25 | 1.56 | 1.56 |
| 43 | V8LQ19L C-1 | KKLLK KWLKL SKKAL KLALS | 3.125 | 3.125 | 3.125 | 3.125 |
| 44 | VBLQ19L C-2 | KKLLK KWLKL SKKAL KLAL | 1.56 | 1.56 | 1.56 | 1.56 |
| 45 | VBLQ18L C-3 | KKLLK KWLKL SKKAL KLA | >6.25 | >6.25 | >6.25 | >8.26 |

Table 8 provides further results for variants which comprise three types of variations in their sequences, demonstrate strong potency against both Pseudomonas aeruginosa (PA) and Staphylococcus aureus (SA).

TABLE 8
Anti-bacterial effects of peptides with triple combinations of modifications

| SEQ | | PA | | SA | |
|---|---|---|---|---|---|
| ID | | MIC | MBC | MIC | MBC |
| NOS | Variant Peptides | (μM) | (μM) | (μM) | (μM) |
| 46 | E1AM2 D-V8L Q19L C-2-NH2 | 1.56 | 1.56 | 1.56 | 1.56 |
| 47 | E1AM2 W72Nal V8L Q19L C-2-NH2 | 1.56 | 1.56 | 1.56 | 1.56 |
| 48 | E1AM2 D-W72Nal V8L Q19L C-2- | 1.56 | 1.56 | 1.56 | 1.56 |
| | NH2 | | | | |

Therefore, in one example, the present disclosure provides a peptide variant E1AM2 Q19L-NH2 having a sequence of KKLLKKWVKLSKKALKLALSL (SEQ ID NO: 27). In another example, the present disclosure provides a peptide variant E1AM2 V8L-NH2 having a sequence of KKLLKKWLKLSKKALKLAQSL (SEQ ID NO: 19). In another example, the present disclosure provides a peptide variant E1AM2 V8L Q19L-NH2 having a sequence of KKLLKKWLKLSKKALKLALSL (SEQ ID NO: 39). In another example, the present disclosure peptide variant E1AM2 V8L Q19L C-2-NH2 having a sequence of provides a KKLLKKWLKLSKKALKLAL (SEQ ID NO: 44). In another example, the present disclosure provides a peptide variant E1AM2 W7 (2-Nal) V8L Q19L C-2-NH2 having a sequence of KKLLKK (2-Nal) LKLSKKALKLAL (SEQ ID NO: 47).

In some examples, the peptide as disclosed herein further comprises a partial sequence from ENO3. This sequence is different from the peptide defined in the claims and disclosure. The partial sequence from ENO3 can be directly or indirectly linked to the peptide sequence as described herein, at the N-terminus or the C-terminus of the sequences as described herein. In a specific example, the ENO3 has an amino acid sequence of SEQ ID NO: 47.

In one example the peptides disclosed herein are ≤21 amino acids long or between 15 to 21 amino acids long. In some examples, the peptides as disclosed herein are amidated at the C-terminus; in some examples, the peptides as disclosed herein are acetylated at the N-terminus. Such modifications can improve the stability of the peptides, compared to unmodified peptides against proteolysis. In one example, the anti-bacterial potency of untagged (E1AM2), N-acetylated (Ac-E1AM2), C-amidated (E1AM2-NH2) and double tagged (Ac-E1AM2-NH2) variants is tested against Pseudomonas aeruginosa (PA) using CLSI guidelines. As can be seen from Table 9 below, all tested variant peptides demonstrate strong potency against bacteria.

TABLE 9
Effects of terminal modifications of E1AM2
against Pseudomonas aeruginosa (PA)

| SEQ | | | |
|---|---|---|---|
| ID | | MIC | MBG |
| NOS | PA | (μM) | (μM) |
| 3 | AC-E1AM2 | 25 | 25 |
| 4 | Ac-E1AM2-NH2 | 12.5 | 12.5 |
| 2 | E1AM2 | 12.5 | 25 |
| 2 | E1AM2-NH2 | 3.125 | 6.25 |

Chronic wounds mostly present mixed infections where numerous pathogens colonize together. In some further examples, the peptide variants provided herein show effective bacterial killing with mixed pathogen environment. Table 10 below provides the minimum inhibitory concentration (MIC) and minimum bactericidal concentration (MBC) values for exemplary peptide variant E1AM2 in a mixed bacterial cell culture. E1AM2 and antibiotic gentamicin could kill mixed infection at 6.25 μM, as opposed to pexiganan and other antibiotics which were required at higher dosages. Although the antibiotic gentamicin showed equal efficacy to E1AM2, it induced resistance development in bacteria. Thus, the peptides disclosed exhibit maximum anti-bacterial efficacy in a mixed bacterial infection model mimicking a chronic wound environment.

TABLE 10
MICs (μM) and MBC (μM) of peptides
in a mixed bacterial infection

| SEQ | | | |
|---|---|---|---|
| ID | Mixed | MIC | MBC |
| NOS | Infection | (μM) | (μM) |
| 54 | Pexiganan | 6.25 | 12.5 |
| 2 | E1AM2-NH2 | 6.25 | 6.25 |
| — | Gentamicin | 3.125 | 6.26 |
| — | Piperacillin | 25 | 25 |
| — | Ciprofloxacin | 25 | 25 |
| — | Cefepime | 12.5 | 12.5 |
| — | Colistin | >100 | >100 |

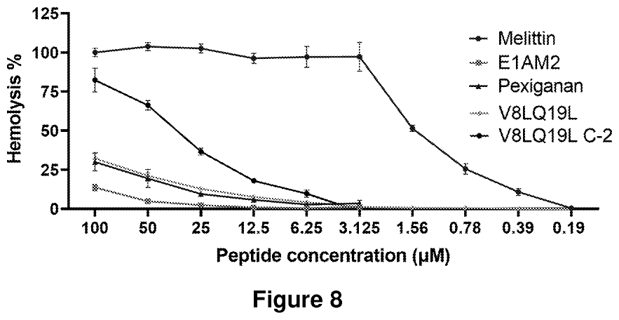

In another example, the anti-bacterial peptides as described herein are non-cytotoxic. In a further example, the anti-bacterial peptides as described herein are non-hemolytic. As shown in FIG. 8, the peptide variants do not cause hemolysis in human red blood cells (hRBCs) under therapeutic concentrations.

In another aspect, the present disclosure provides a composition comprising one or more of the (anti-bacterial) peptides as described herein. In another aspect, the present disclosure provides a pharmaceutical composition comprising the peptide as described herein, or the composition as described herein, and a pharmaceutically acceptable carrier or excipient. A pharmaceutically acceptable acid salt can be formed by reaction of the compound with a suitable acid such as acetic, benzoic, citric, formic, fumaric, hydrobromic, hydrochloric, maleic, methanesulfonic, naphthalenesulfonic, nitric, oxalic, p-toluenesulfonic, phosphoric, succinic, sulfuric, tartaric or trifluoroacetic acid.

In some examples, the peptide, the composition, or the pharmaceutical composition, is formulated in the form of a solution, a lyophilized powder, a gel, a lotion, a cream, an ointment, an aerosol. Formulation of the peptide can be in the form of cream/gel (for dry wound treatment) by integration into delivery systems such as homopolymer, PEG; copolymer, CMC; self-assembly peptide system; or heparin. For wet wound treatment, the peptide can be formulated in the form of wound dressing scaffold, for example, by means of natural polymers such as alginate, hyaluronic acid, and collagen. In some examples, the peptide, the composition, or the pharmaceutical composition is formulated for topical application, such as an eye drop, an ear drop, a nasal drop, a nasal spray or an oral spray. In further examples, the peptide, the composition, or the pharmaceutical composition is embedded in a hydrogel, a polymer, a nanofiber, or attached to a nanoparticle for delivery. Delivery systems such as hydrogels or poly(lactic-co-glycolic) acid (PLGA) nanoparticles can increase the bioavailability and stability of the peptides. Formulations according to the invention intended for topical administration can contain appropriate conventional additives, including, for example, preservatives, solvents to assist drug penetration, and emollients in ointments and creams. Compositions according to the disclosure can also contain one or more steroidal anti-inflammatory agents; for example, betamethasone or dexamethasone. Compositions according to the present disclosure can also comprise one or more non-steroidal anti-inflammatory agents, such as diclofenac, ibuprofen, ketoprofen, salicylic acid. The compositions of the present invention can be for topical application, e.g. a composition of the present invention for topical application also comprising one or more non-steroidal anti-inflammatory agent or one or more steroidal anti-inflammatory agents as defined herein. A person skilled in the art would be able to provide suitable carrier or excipients for the manufacture of pharmaceutical composition based on the dosage form or routes of administration needed.

The variant peptides, composition, and pharmaceutical composition of the present disclosure can also be administered topically to the skin, soft and adipose tissue, wound or mucosa, dermally or transdermally. The variant peptides, composition, and pharmaceutical composition of the present disclosure can also be administered directly to the eye or ear, typically in the form of drops of micronized suspension or solution in isotonic, pH-adjusted sterile saline. The compounds can also be inhaled to treat infection of the respiratory tract. Typical inhalers and inhalation formulations can be used.

In another aspect, the present disclosure provides a wound dressing comprising the peptide as described herein, the composition as described herein, or the pharmaceutical composition as described herein. As used herein, the term “wound dressing” refers to a sterile pad applied to a wound to promote healing and protect the wound from further harm. A dressing is designed to be in direct contact with the wound, which may or may not be self-adhesive. In some cases, a wound dressing may be medicated, i.e. comprising one or more medicaments suitable for wound healing, suppression of infection, pain management, or other purposes. Material of a wound dressing differs based on the wound type. For example, the wound dressing can be, and is not limited to an alginate dressing, a hydrocolloid dressing, or a foam dressing. A person skilled in the art would be able to assess the type, shape, and position of the wound to be treated and select suitable material and type of dressing to be used.

In another aspect, the present disclosure provides a nucleic acid composition encoding the peptide as described herein. In a further aspect, the present disclosure provides an expression vector comprising the nucleic acid composition as described herein. In another aspect, the present disclosure provides a host cell comprising one or more of the nucleic acid compositions as described herein, or one or more of the expression vectors as described herein. In one example, the host cell is a mammalian cell, a bacterium, a yeast cell, a fungus cell, or a plant cell.

In one aspect, the present disclosure provides a method of treating an infection in a subject. In one example, the method comprises preventing the recurrence of a bacterial infection in a subject. The recurrence of bacterial infections, alternatively referred to as infection relapse or remission, is predominantly caused by the emergence of resistant strains, a phenomenon known as antimicrobial resistance (AMR). FIG. 6 provides an example of such activity with reference to the V8LQ19L peptide variant, which was shown to be effective in preventing the acquisition of AMR in both Pseudomonas aeruginosa (PA) and Staphylococcus aureus (SA). The method as described herein comprises administering to the subject a pharmaceutically effective amount of the peptide as described herein, the composition as described herein, the pharmaceutical composition as described herein, or applying to the subject a wound dressing as described herein. As used herein, the term “pharmaceutically effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with the disease state (e.g., relieving the infection). A person skilled in the art is able to determine a pharmaceutically effective amount for the peptide, the composition, or the pharmaceutical composition as disclosed herein based on considerations such as disease state, body size, administration frequencies and route.

As shown in FIG. 4, for example, exemplary peptide E1AM2 displays potent in vivo anti-bacterial efficacy in a mouse excisional wound infection model caused by Pseudomonas aeruginosa (PA) infection. E1AM2 at 1x MBC (6.25 μM) concentration showed effective reduction of PA infection at the wound site in contrast to PBS treatment.

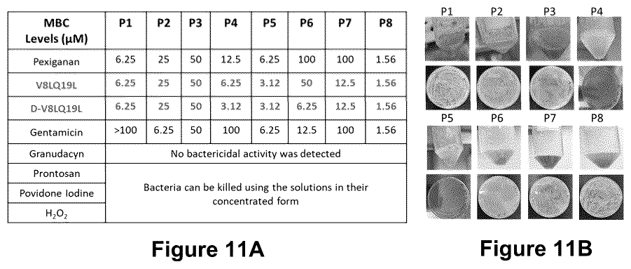

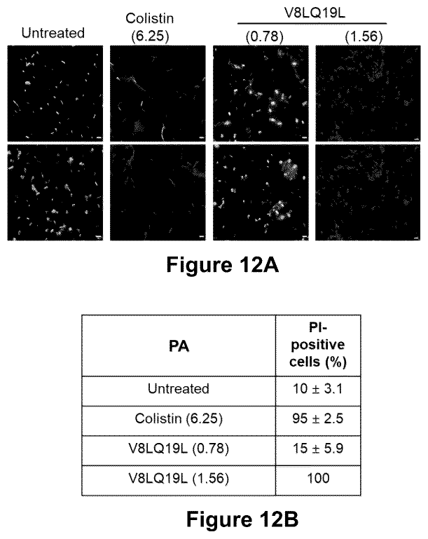

In some examples, the effective bacteria killing concentration range for the variant peptides can be, but is not limited to at least about 1.5 μM, at least about 2 μM, at least about 2.5 μM, at least about 5 μM, at least about 7.5 μM, at least about 10 μM, at least about 12.5 μM, at least about 15 μM, at least about 15 μM, at least about 17.5 μM, at least about 20 μM, at least about 25 μM, at least about 35 μM, or at least about 45 μM. In one example, the effective biofilm disruption concentration ranges from about 1.5 μM-25 μM or 1.5 μM-50 μM. In another example, the concentration is between about 40 μM to 50 μM. For example, as demonstrated in FIG. 11, in one instance involving infected tissue samples from patients with Diabetic Foot Ulcers (DFU), V8LQ19L was shown to work at a concentration of 50 μM to eliminate over 99% of the bacteria.

The bactericidal abilities of the variant peptides are not only effective, but also fast-acting. Based on a time-kill assay, variant peptides E1AM2, V8L, Q19L and V8LQ19L at their 1X minimum bactericidal concentration (MBC) levels could kill both Pseudomonas aeruginosa (PA) and Staphylococcus aureus (SA) strains within minutes. In particular, V8LQ19L exhibited killing within seconds against both Pseudomonas aeruginosa (PA) and Staphylococcus aureus (SA). Pexiganan as a control took the longest time to kill >99% PA. The killing kinetics are detailed in Table 11 below.

TABLE 11
Killing kinetics of E1AM2 and variants against Pseudomonas aeruginosa
(PA) and Staphylococcus aureus (SA) strains compared to pexiganan.

| | | | Time kill | | | | Time kill |
|---|---|---|---|---|---|---|---|
| | | MBC | PA (min) | | | MBC | SA (min) |
| SEQ | | (μM) | >99% | SEQ | | (μM) | >99% |
| ID NOs | Peptides | 1X | killing | ID NOs | Peptides | 1X | killing |
| 54 | Pexiganan | 6.25 | >5-15 | 54 | Pexiganan | 12.5 | 1 |
| 2 | E1AM2 | 6.25 | 1 | 2 | E1AM2 | 12.5 | 1 |
| 19 | V8L | 3.125 | 1* | 19 | V8L | 3.125 | >30-60 |
| 27 | Q19L | 6.25 | >1.5 | 27 | Q19L | 3.125 | 1 |
| 39 | VBLQ19L | 1.56 | 1* | 39 | V8LQ19L | 1.56 | 1* |

*Indicates killing within seconds

In some examples, the infection is a mixed infection. In some examples, the infection is a mixed infection comprising Pseudomonas aeruginosa. In some examples, the infection is a mixed infection comprising Staphylococcus aureus. In one example, the infection is a bacterial infection. In another example, the subject may further comprise a fungal infection, a protist infection, or a combination thereof. In some examples, the infection can be caused by one or more of the following microorganisms: Salmonella typhimurium, Pseudomonas aeruginosa, Staphylococcus aureus, Enterococcus faecalis, Klebsiella pneumoniae, Actinetobacter baumannii, Escherichia coli, Enterobactor cloacae, Serratia marcescens, Fusobacterium nucleatum, Porphyromonas gingivalis, Prevotella intermedia, Streptococcus sanguinis, or Lactobacillus casei. The peptides described herein can also be used for the treatment of bacteria including but are not limited to: Actinetobacter baumannii, Acinetobacter johnsonii, Bacillus anthracis, Bacillus cereus, Chlamydia trachomatis, Clostridium perfringens, Corynebacterium spp., Cutibacterium acnes, Enterobacteriaceae, Enterobacter spp., Enterobacter cloacae, Enterococcus faecalis, Escherichia coli, Fusobacterium nucleatum, Haemophilus influenzae, Klebsiella pneumoniae, Lactobacillus casei, Prevotella intermedia, Porphyromonas gingivalis, Propionibacterium acnes, Proteus spp., Pseudomonas aeruginosa, Serratia spp., Serratia marcescens, Staphylococcus aureus, Staphylococcus epidermidis, Staphylococcus hominis, Staphylococcus saprophyticus, Staphylococcus warneri, Streptococcus agalactiae, Streptococcus mitis, Streptococcus pneumoniae, Streptococcus pyogenes, Streptococcus sanguinis, Staphylococcus epidermidis, Streptococcus agalactiae, Streptococcus pneumoniae, Streptococcus pyogenes, Haemophilus sp., Neisseria sp., Legionella sp., Chlamydia sp., and Moraxella catarrhalis.

In another example, the infection is a chronic infection, or a nosocomial infection. In a further example, the infection is an infection caused by a drug resistant microorganism. In a further example, the infection is caused by a multi-drug resistant microorganism. In some examples, the infection is resistant to one or more anti-bacterial agents, such as, but are not limited to: vancomycin, ampicillin, gentamicin, kanamycin A, neomycin B, neomycin C, neomycin E, amikacin, tobramycin, dibekacin, sisomicin, netilmicin, streptomycin, plazomicin, ticarcillin, pefloxacin, ceftriaxone, and methicillin. FIGS. 6A and 6B provide examples of drug resistant bacterial strains, which respond to the treatment of the exemplary peptides E1AM2 and Q19L described herein even after 28 days of prolonged treatment.

In one example, the present disclosure provides the use of the peptide as described herein, the composition as described herein, or the pharmaceutical composition as described herein in the manufacture of a medicament for treating an infection in a subject.

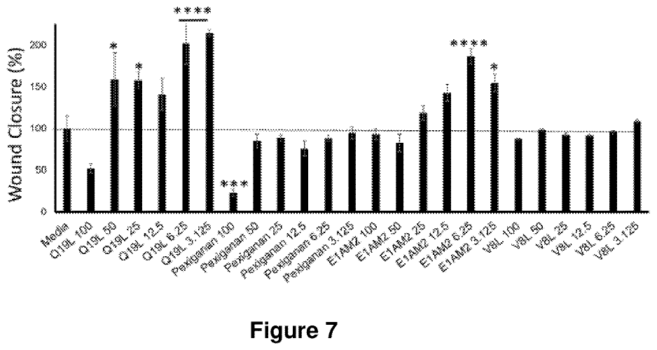

In one aspect, the present disclosure provides a method of promoting wound healing and tissue regeneration in a subject. The method comprises administering to the subject a pharmaceutically effective amount of the peptide as described herein, the composition as described herein, the pharmaceutical composition as described herein or applying to the subject a wound dressing as described herein. As shown in FIG. 7, exemplary peptide variants E1AM2 and Q19L increase keratinocyte migration in 2D scratch wound model, indicating significant wound-healing ability apart from anti-bacterial efficacy as wound healing is directly associated with the migration speed of keratinocytes.

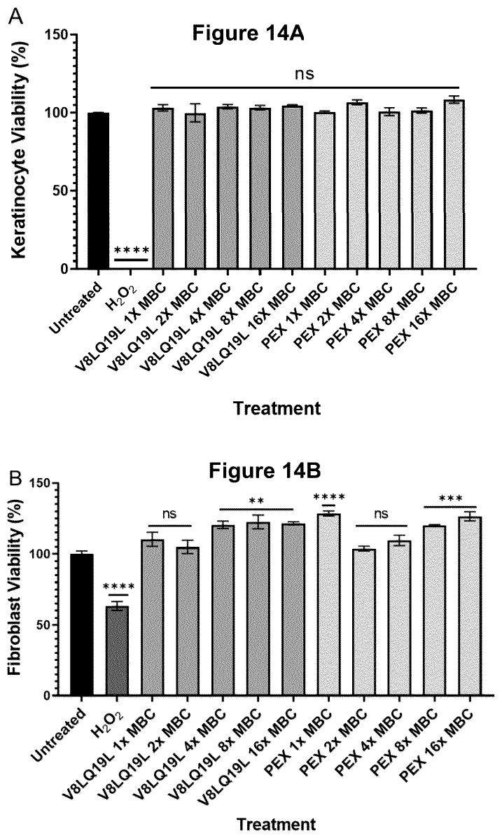

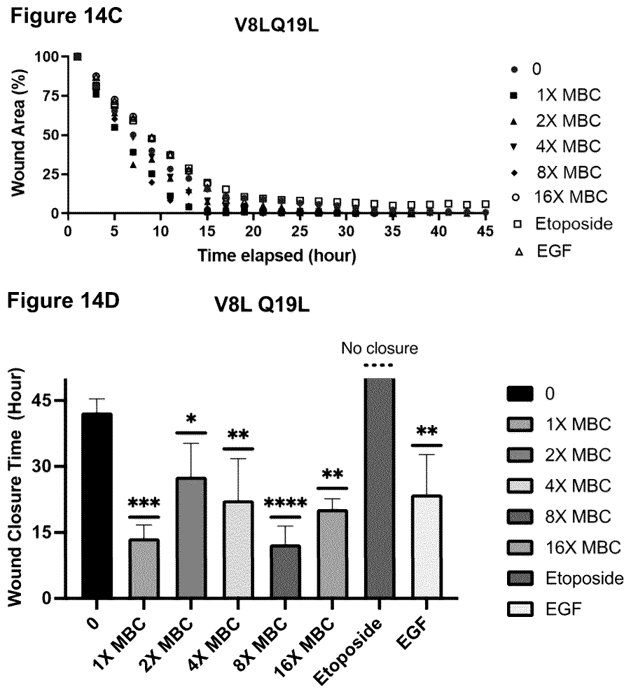

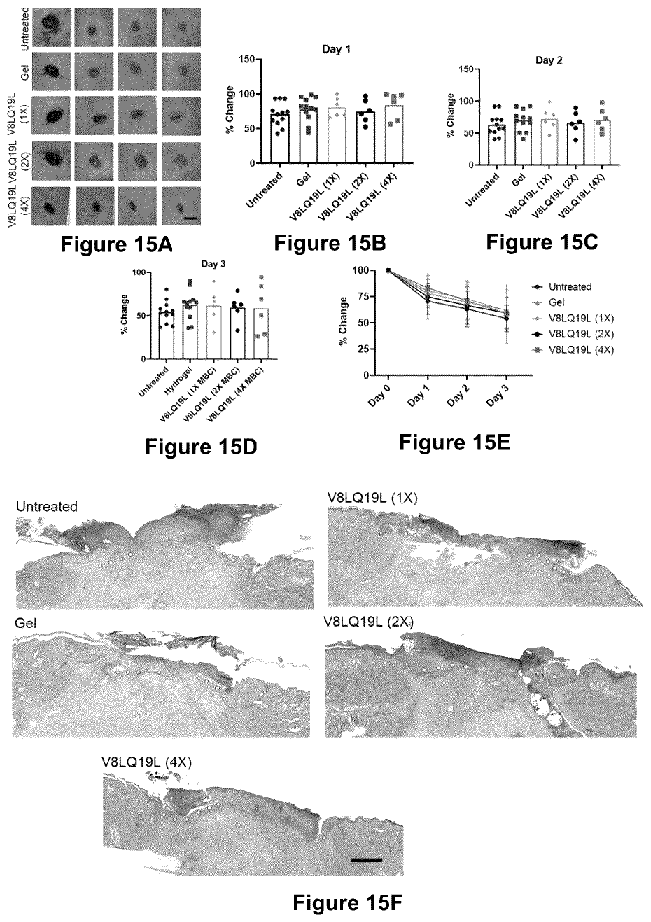

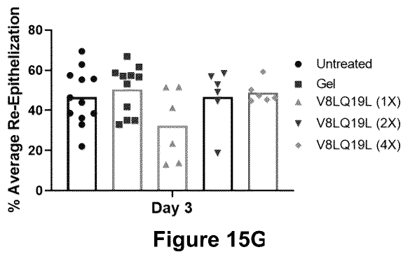

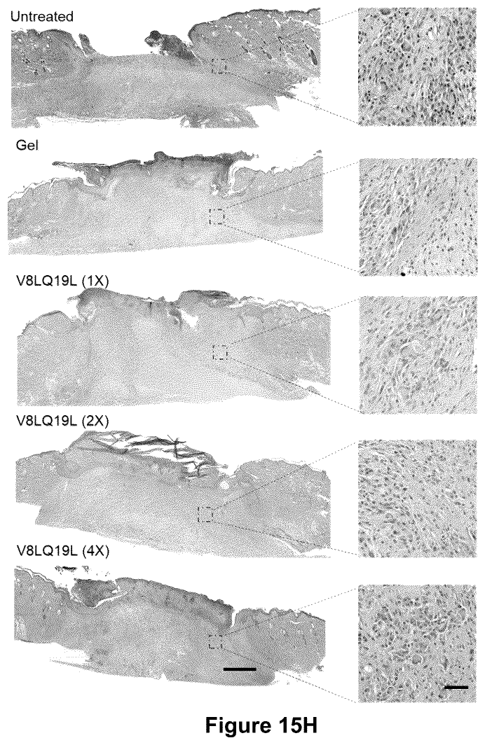

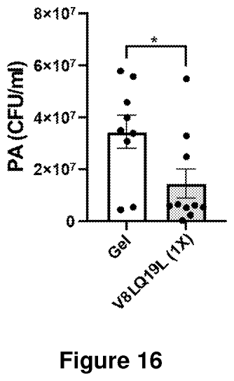

In some examples, the effective wound healing concentration range for the variant peptides can be, but is not limited to at least about 3 μM, at least about 3.5 μM, at least about 4 μM, at least about 4.5 μM, at least about 5 μM, at least about 5.5 μM, at least about 6 μM, at least about 6.5 μM, at least about 8 μM, at least about 10 μM, at least about 12 μM, or at least about 12.5 μM. In one example, the effective biofilm disruption concentration ranges from about 3 μM-15 μM. For example, as illustrated by FIG. 14, the in vitro data reveal an increase in wound healing, evidenced by significantly faster wound closure, even when applying concentrations up to 16 times the MBC levels of the peptide. In vivo results support that there is no impairment of wound healing at concentrations up to 4 times the MBC (FIG. 15), indicating that the peptides as disclosed herein do not hinder the process, even at elevated concentrations.

In one example, the wound is an infected wound. In a further example, the wound is suffering from a chronic infection, a healthcare-associated infection, or a nosocomial infection. In one example, the wound is infected by one or more microorganisms. In another example, the wound is infected by one or more bacteria. In some examples, the wound is infected by one or more Gram-positive and/or Gram-negative bacteria strains. In some examples, the wound is infected by bacteria including, but are not limited to: Salmonella typhimurium, Pseudomonas aeruginosa, Staphylococcus aureus, Enterococcus faecalis, Klebsiella pneumoniae, Actinetobacter baumannii, Escherichia coli, Enterobactor cloacae, Serratia marcescens, Fusobacterium nucleatum, Porphyromonas gingivalis, Prevotella intermedia, Streptococcus sanguinis, or Lactobacillus casei. In some examples, the bacterium in the wound is resistant to one or more anti-bacterial agents, such as, but are not limited to: amikacin, amoxicillin, ampicillin, azithromycin, aztreonam, cefepime, ceftatroline, ceftazidime, ceftriaxone, ciprofloxacin, clarithromycin, clindamycin, colistin, daptomycin, dibekacin, doripenem, doxycycline, ertapenem, erythromycin, fusidic acid, gentamicin, kanamycin A, levofloxacin, linezolid, methicillin, meropenem, minocycline, neomycin B, neomycin C, neomycin E, netilmicin, oxacillin, pefloxacin, piperacillin, plazomicin, rifampicin, sisomicin, streptomycin, tetracycline, ticarcillin, tigecycline, tobramycin, vancomycin.

In one example, the present disclosure provides the use of the peptide as described herein, the composition as described herein, the pharmaceutical composition as described herein, or the wound dressing as described herein in the manufacture of a medicament for promoting wound healing and tissue regeneration in a subject.

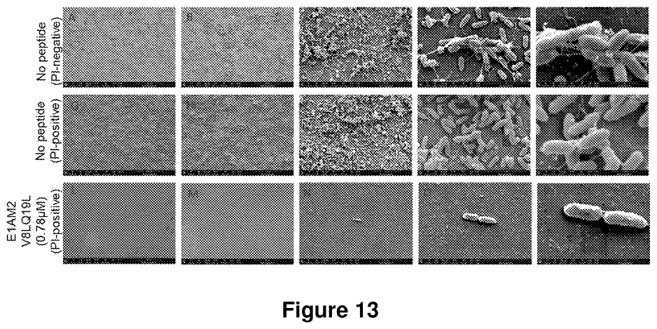

In one aspect, the present disclosure provides a method of reducing biofilm in a subject. In another aspect, the present disclosure provides a method of preventing formation of biofilm in a subject. For example, FIG. 13 illustrates that in the presence of the peptide V8LQ19L, Pseudomonas aeruginosa (PA) either fails to form biofilms or experiences complete disruption of existing biofilms, with no remnants observed, in stark contrast to the control group without the peptide V8LQ19L. The method comprises administering to the subject a pharmaceutically effective amount of the peptide as described herein, the composition as described herein, the pharmaceutical composition as described herein or applying to the subject a wound dressing as described herein. FIG. 3 shows effective disruption of the biofilm formed by Pseudomonas aeruginosa (PA), which is a major bacteria strain found in biofilms in chronic wounds. Upon treatment with an exemplary peptide variant E1AM2, the biofilm is disrupted with extracellular polysaccharides (EPS) formed by PA no longer present under scanning electron microscope (SEM) imaging.

In one example, the biofilm comprises one or more Gram-positive or Gram-negative bacteria strains. In a further example, bacteria present in the biofilm can be, and are not limited to: Actinetobacter baumannii, Acinetobacter johnsonii, Bacillus anthracis, Bacillus cereus, Chlamydia trachomatis, Clostridium perfringens, Corynebacterium spp., Cutibacterium acnes, Enterobacteriaceae, Enterobacter spp., Enterobacter cloacae, Enterococcus faecalis, Escherichia coli, Fusobacterium nucleatum, Haemophilus influenzae, Klebsiella pneumoniae, Lactobacillus casei, Prevotella intermedia, Porphyromonas gingivalis, Propionibacterium acnes, Proteus spp., Pseudomonas aeruginosa, Serratia spp., Serratia marcescens, Staphylococcus aureus, Staphylococcus epidermidis, Staphylococcus hominis, Staphylococcus saprophyticus, Staphylococcus warneri, Streptococcus agalactiae, Streptococcus mitis, Streptococcus pneumoniae, Streptococcus pyogenes and Streptococcus sanguinis. In one example, the bacteria present in the biofilm is resistant to one or more anti-bacterial agents. In a further example, the one or more anti-bacterial agents can be, but are not limited to amikacin, amoxicillin, ampicillin, azithromycin, aztreonam, cefepime, ceftatroline, ceftazidime, ceftriaxone, ciprofloxacin, clarithromycin, clindamycin, colistin, daptomycin, dibekacin, doripenem, doxycycline, ertapenem, erythromycin, fusidic acid, gentamicin, kanamycin A, levofloxacin, linezolid, methicillin, meropenem, minocycline, neomycin B, neomycin C, neomycin E, netilmicin, oxacillin, pefloxacin, piperacillin, plazomicin, rifampicin, sisomicin, streptomycin, tetracycline, ticarcillin, tigecycline, tobramycin, vancomycin.

In some examples, the effective biofilm disruption concentration range for the variant peptides can be, but is not limited to at least about 1 μM, at least about 1.5 μM, at least about 2 μM, at least about 2.5 μM, at least about 5 μM, at least about 7.5 μM, at least about 10 μM, at least about 12.5 μM, at least about 15 μM, at least about 15 μM, at least about 17.5 μM, at least about 20 μM, at least about 25 μM, at least about 35 μMm, or at least about 45 μM. In one example, the effective biofilm disruption concentration ranges from about 1.5 μM-25 μM or 1.5 μM-50 μM. In another example, a concentration of 50 μM was shown to be effective.

In some examples, the present disclosure provides the use of the peptide as described herein, the composition as described herein, the pharmaceutical composition as described herein or the wound dressing as described herein in the manufacture of a medicament for reducing biofilm in a subject or preventing the formation of biofilm in a subject.

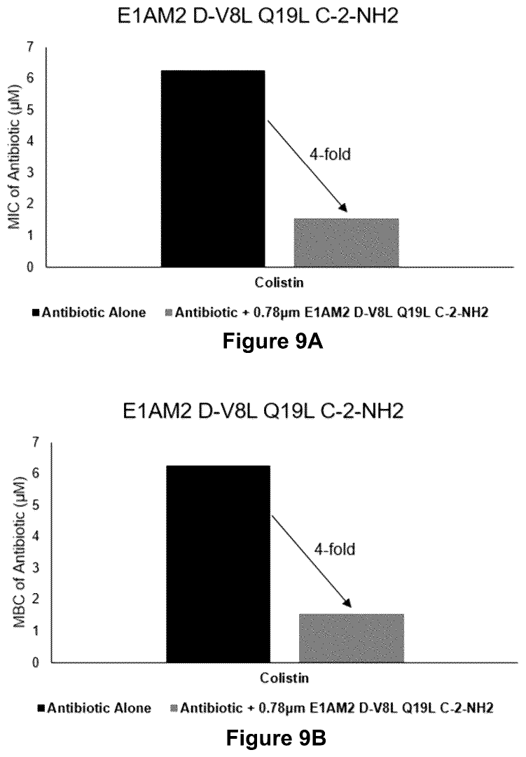

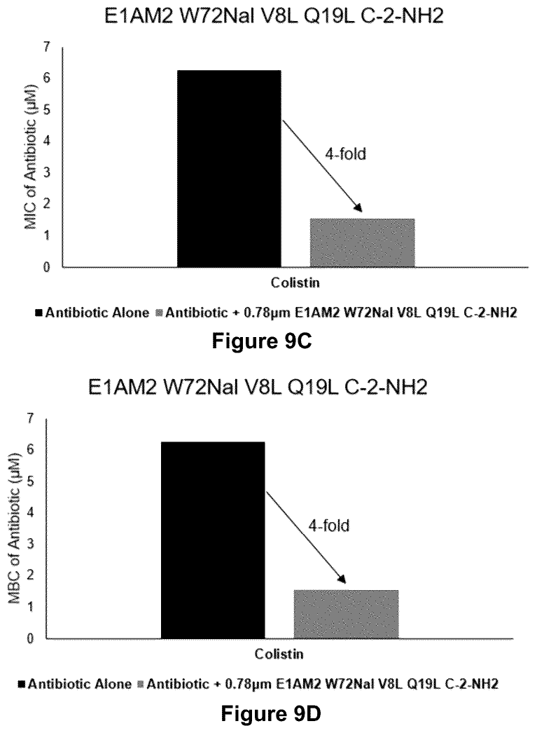

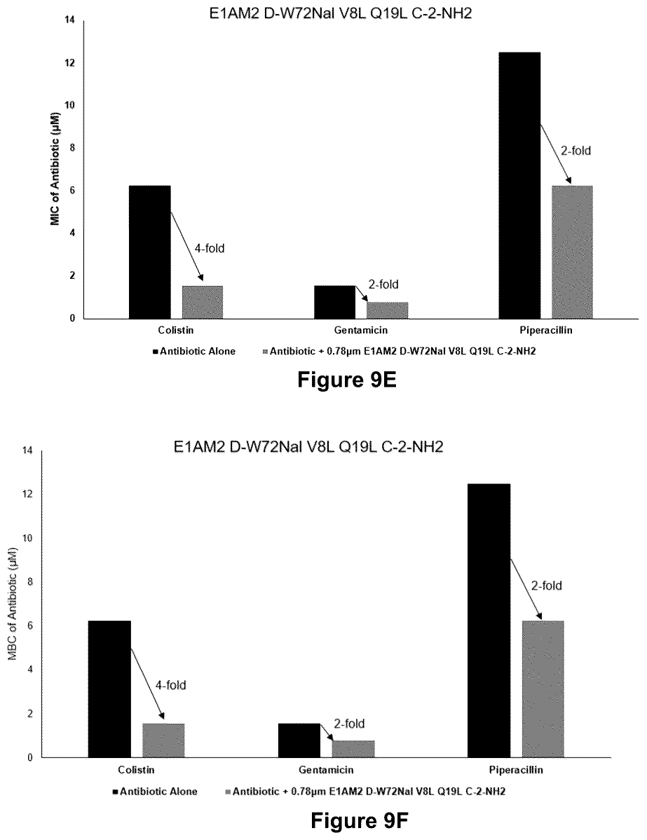

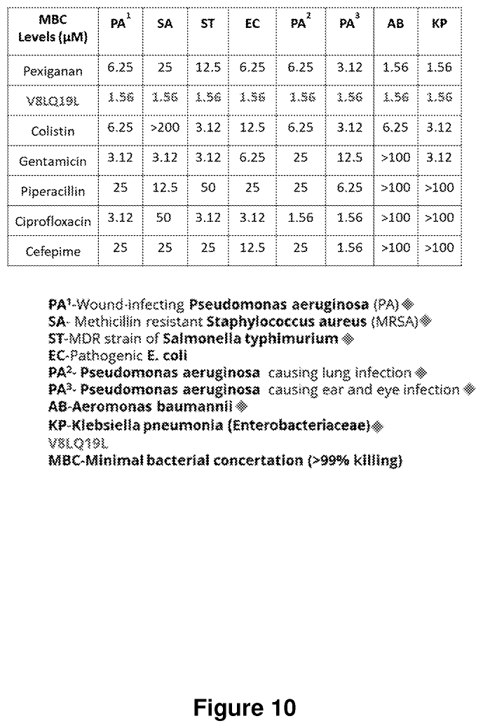

In some examples, the methods described herein further comprises administering an antibiotic agent to the subject. As shown in FIG. 9, the combination of peptide and antibiotic is able to reduce minimum inhibitory concentration (MIC) and minimum bactericidal concentration (MBC) values of the antibiotic, thus potentiating/augmenting the antibiotics. FIG. 9 demonstrates the significant synergistic effects between the variant peptides with exemplary antibiotics such as colistin, gentamicin, and piperacillin.

The antibiotic agent can be used in combination with the variant peptides can be, but is not limited to: gentamicin ((3R,4R,5R)-2-{[(1S,2S,3R,4S,6R)-4,6-diamino-3-{[(2R,3R,6S)-3-amino-6-[(1R)-1-(methylamino)ethyl]oxan-2-yl]oxy}-2-hydroxycyclohexyl]oxy}-5-methyl-4-(methylamino) oxane-3,5-diol), colistin (N-(4-amino-1-(1-(4-amino-1-oxo-1-(3,12,23-tris(2-aminoethyl)-20-(1-hydroxyethyl)-6,9-diisobutyl-2,5,8,11,14,19,22-heptaoxo-1,4,7,10,13,18-hexaazacyclotricosan-15-ylamino)butan-2-ylamino)-3-hydroxybutan-2-ylamino)-1-oxobutan-2-yl)-N,5-dimethylheptanamide) piperacillin ((2S,5R,6R)-6-{[(2R)-2-[(4-ethyl-2,3-dioxo-piperazine-1-carbonyl)amino]-2-phenyl-acetyl]amino}-3,3-dimethyl-7-oxo-4-thia-1-azabicyclo[3.2.0]heptane-2-carboxylic acid), kanamycin A (2-(aminomethyl)-6-[4,6-diamino-3-[4-amino-3,5-dihydroxy-6-(hydroxymethyl)tetrahydropyran-2-yl]oxy-2-hydroxy-cyclohexoxy]-tetrahydropyran-3,4,5-triol), kanamycin B ((2S,3R,4S,5S,6R)-4-amino-2-{[(2S,3R,4S,6R)-4,6-diamino-3-{[(2R,3R,4R,5S,6R)-3-amino-6-(aminomethyl)-4,5-dihydroxyoxan-2-yl]oxy}-2-hydroxycyclohexyl]oxy}-6-(hydroxymethyl) oxane-3,5-diol), nalidixic acid (1-Ethyl-7-methyl-4-oxo-[1,8]naphthyridine-3-carboxylic acid), ampicillin ((2S,5R,6R)-6-([(2R)-2-Amino-2-phenylacetyl]amino)-3,3-dimethyl-7-oxo-4-thia-1-azabicyclo[3.2.0]heptane-2-carboxylic acid), vancomycin ((1S,2R,18R,19R,22S,25R,28R,40S)-48-{[(2S,3R,4S,5S,6R)-3-{[(2S,4S,5S,6S)-4-amino-5-hydroxy-4,6-dimethyloxan-2-yl]oxy}-4,5-dihydroxy-6-(hydroxymethyl) oxan-2-yl]oxy}-22-(carbamoylmethyl)-5,15-dichloro-2,18,32,35,37-pentahydroxy-19-[(2R)-4-methyl-2-(methylamino) pentanamido]-20,23,26,42,44-pentaoxo-7,13-dioxa-21,24,27,41,43-pentaazaoctacyclo[26.14.2.2^{3,6}.2^{14,17}.1^{8,12}.1^{29,33}.0^{10,25}.0^{34,39}]pentaconta-3,5,8 (48),9,11,14,16,29 (45),30,32,34,36,38,46,49-pentadecaene-40-carboxylic acid), linezolid ((S)-N-({3-[3-fluoro-4-(morpholin-4-yl)phenyl]-2-oxo-1,3-oxazolidin-5-yl}methyl) acetamide) and ciprofloxacin (1-cyclopropyl-6-fluoro-4-oxo-7-(piperazin-1-yl)-quinoline-3-carboxylic acid).

In one example, the peptide, the composition, the pharmaceutical composition, or the wound dressing, is administered/applied together with the one or more antibiotic agents. In another example, the peptide, the composition, the pharmaceutical composition, or the wound dressing, is administered/applied separately with the one or more antibiotic agents.

In some examples, the subject to be treated is, has been, or is at risk of being diagnosed to be obese or overweight. In some examples, the subject to be treated is, has been, or is at risk of being diagnosed to be diabetic. In some examples, the subject to be treated has diabetic foot ulcer and/or diabetic foot infections. In some examples, the subject to be treated has bacterial infection in the lungs, eyes, ears, nose, pharynx, larynx, soft tissue or skin.

Further provided in Table 12 below are the minimum inhibitory concentration (MIC) and minimum bactericidal concentration (MBC) of the peptide variants in killing bacteria involved in lung, and ear-eye infections, indicating broad-spectrum activities besides chronic wound healing. Bacteria strains involved in lung infection in cystic fibrosis patients (PA-BAA 2110) and ear-eye infections (PA-CRM 9027) are treated with exemplary variant peptides, such as E1AM2 and Q19L. Both variants could successfully clear the pathogens strains with MBC of 6.25 μM. Thus, the variant peptides as disclosed herein have broad-spectrum applications against various types of infections.

TABLE 12
MICs (μM) and MBC (μM) of variant peptides, pexiganan and
antibiotics against Pseudomonas aeruginosa (PA) strains

| SEQ | PA | MIC | MBC | PA | MIC | MBC |
|---|---|---|---|---|---|---|
| ID NOs | BAA-2110 | (μM) | (μM) | CRM-9027 | (μM) | (μM) |
| 54 | Pexiganan | 3.125 | 6.25 | Pexiganan | 3.125 | 3.125 |
| 2 | E1AM2 | 3.125 | 6.25 | E1AM2 | 3.125 | 3.125 |
| 27 | Q19L | 6.25 | 6.25 | Q19L | 6.25 | 6.25 |
| — | Colistin | 6.25 | 6.25 | Colistin | 3.125 | 3.125 |
| — | Gentamicin | 25 | 25 | Gentamicin | 6.25 | 12.5 |
| — | Piperacillin | 12.5 | 25 | Piperacillin | 6.25 | 6.25 |
| — | Cipro- | 1.56 | 1.56 | Cipro- | 1.56 | 1.56 |
| | floxacin | | | floxacin | | |
| — | Cefepime | 12.5 | 25 | Cefepime | 1.56 | 1.56 |

Variant peptides of the present disclosure can therefore be used for the treatment or control of nasal carriage of pathogenic bacteria, in particular colonization of the nasospharynx by such pathogens, which is also useful in prophylaxis of recurrent acute bacterial infections.

In some further examples, the mode of administration for the peptide as described herein, the composition as described herein, and the pharmaceutical composition as described herein can be, but is not limited to intramuscular, topical, transdermal, and intravenous administration. As demonstrated herein, for example, in FIGS. 6A and 6B, the infection does not develop resistance to the peptide as described herein, the composition as described herein, and the pharmaceutical composition as described herein, or the wound dressing of as described herein after administration of the same, for example, over 28 days.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

It should further be appreciated that the exemplary embodiments are only examples, and are not intended to limit the scope, applicability, dimensions, or configuration of the invention in any way. Rather, the foregoing detailed description will provide those skilled in the art with a convenient road map for implementing an exemplary embodiment of the invention, it being understood that various changes may be made in the function and arrangement of elements and method of fabrication described in an exemplary embodiment without departing from the scope of the invention as set forth in the appended claims.

### Examples

### Methods

### Bacterial Strains

Pseudomonas aeruginosa (ATCC 14213, PA), and methicillin resistant Staphylococcus aureus (BAA1717-USA 300, SA) were obtained from The American Type Culture Collection (ATCC).

### Peptide Synthesis

Short-length AMPs were then synthesized to >98% purity commercially by Mimotopes Pty Ltd by solid phase peptide synthesis (SPSS).

### Anti-Bacterial Assay

Examination of the potency of modified short peptides against PA and SA strains was performed by using the broth microdilution protocol from the Clinical and Laboratory Standards Institute (CLSI) guidelines (CLSI 2012) as described. Briefly, five freshly plated bacteria colonies were grown in cation adjusted MH2 broth (BD Biosciences) in a shaker incubator at 37° C., 220 RPM. Cells were grown to an optical density (OD600) of ~0.2 measured using a Microplate Spectrophotometer (Molecular Devices Spectra Max Plus). 100 μL of microbial culture containing ~5×10⁵ CFU/mL of bacteria in the respective broths were introduced into each well containing 100 μL of peptides and incubated overnight at 37° C. Next, to check the bactericidal effects, 100 μL from each well was transferred and spread on sterile MH2 agar plates. The plates were incubated overnight at 37° C. and the number of colonies forming units (CFU) were calculated the following day. Readings of bacteria grown in media without any treatments were normalized to 100% as media control. For the mixed infection study, three freshly plated bacteria colonies each from PA, SA and ST plates were grown together in cation adjusted MH2 broth and proceeded as described above. MIC and MBC values were defined as the lowest peptide concentration (μM) required to either inhibit (MIC) or kill (MBC) ≥99% bacteria compared to the media control.

### Antimicrobial Resistance Evolution Assay

We assessed resistance development of Pseudomonas aeruginosa (PA) and Staphylococcus aureus (SA) strains by sequential passaging in the presence of sub-inhibitory concentrations of chronic wound relevant antibiotics (colistin, ciprofloxacin, gentamicin, piperacillin, and cefepime) and our modified short peptide, E1AM2. Briefly, sequential passaging of PA, and SA cells was done with sub-inhibitory concentrations of antibiotics or E1AM2 up to 4 weeks. The cells grown in those concentrations of antibiotics or peptides were diluted to A600 nm (OD600) of ~0.2 and serially passaged daily with the same concentrations at 37° C. with agitation at 24 h intervals. Experiment was discontinued once the MIC reached >100 μM or increased by >4-fold from the initial start point (day 1) or when the bacterial cells failed to grow any further.

### Cell Viability Assay

CyQUANT™ Direct Cell Proliferation Assay (ThermoFisher Scientific, Catalogue) was performed as per manufacturer's instructions to determine the safety of the peptides to human keratinocytes. Peptide E1AM2 was incubated from sub-inhibitory concentrations to 100 μM with the keratinocytes (HaCaT) for 48 hours. The fluorescence intensity and number of stained cells were determined using the LionHeart FX Automated Microscope (BioTek, VT, USA).

### 2D Scratch Assay

Wound areas of the human keratinocyte culture were assessed in presence of peptides in a 2D scratch assay at the 0 hour, and 48-hour time points by applying an ImageJ plugin, ‘Wound Healing Size Tool’ on brightfield images. Quantification of wound width was calculated by obtaining the averaged Euclidean distance between each paired pixel at the lateral wound edges. The change in wound area over the course of 48 hours was used as a measure of the migration speed of keratinocytes.

### Live-Dead Staining of Bacteria

PA was treated with E1AM2 for 24 hours followed by live-dead saining in accordance to the manufacturers' instruction (LIVE/DEAD™ BacLight™ Bacterial Viability Kit, for microscopy & quantitative assays).

### Time-Kill Assay

Pseudomonas aeruginosa (PA) and Staphylococcus aureus (SA) strains were grown as per CLSI guidelines. AMPs were added to the bacterial cultures at their 1X MBC concentrations. Upon treatment, the bacterial cultures were enumerated for viable counts immediately within 1 min, 5 min, 15 min, 30 min and 60 min post-treatments.

### Microscopy

Using the inverted BioTek Lionheart LX Automated Microscope, brightfield images were obtained at 4× magnification to observe the wound area at timepoints 0 (before treatment) and 48 hours-post treatment. Microscopy settings were maintained as focus offset (781.3 μm), illumination (2), integration Time (21) with no gain. To cover the entire well, montage images of four rows by three columns were stitched together by enabling the “image stitching” function and images were saved in tiff-format.

### Hemolysis Assay

Examination of hemolytic activity of ultra-short peptides was performed by isolating the red blood cells (RBCs) from human blood samples by centrifugation (1000 g, 4° C., 10 min) and resuspension in 27 mL PBS. This washing step was repeated four times before the RBCs were diluted to a 10% (v/v) suspension. Diluted RBC suspension (50 μL) was aliquoted into each well of a round-bottom 96-well clear plate containing peptides ranging from 3.125 μM to 100 μM increasing in 2-fold increments. Hemolytic properties of E1AM2 and pexiganan were evaluated with melittin used as a positive control, and PBS as a negative control. The plates were incubated at 37° C., for 30 min and then centrifuged (1258 g, 4° C., 6 min). 75 μL of supernatant were transferred into a flat-bottom clear 96-well plate and the OD576 nm was read. Readings of the melittin sample at 3.125 μM were normalized to 100%.

### Murine Excisional Wound Model

The murine wound infection model was carried out as described in 4 with minor modifications. Before infection, PA strain was diluted to an OD ~0.5, and normalized to 1-3×10⁷ CFU/ml. A 6 mm biopsy punch (Integra Miltex, New York, USA) was used to create a full-thickness wound, and an inoculum of ~10⁵ CFU of the bacteria in a 5 μl volume was applied. The wound was sealed with a Finn chamber on a Scanpor tape (Smart Practice, Phoenix, AZ, USA), and the chamber was fixed to the skin via Tegaderm patch (3M™, Minnesota, USA). After 24 h post-infection, the Finn chambers were removed and either E1AM2 (6.125 μM) or PBS were added, prior to re-sealing with the tegaderm patch for another 24 hours The mice were euthanized and a 1 cm×1 cm squared piece of skin surrounding the wound site was excised and collected in sterile 1×PBS. Enumeration of the bacterial count was done as described.

### Statistical Analysis

Results were represented as means±SEM. Student's t-test was used to determine differences in means between two groups. The p value was calculated using ANOVA for multiple comparisons with corrections and p<0.05 being considered as significant.

### Comparative Results

The modified variant peptides described herein are fast-acting, non-cytotoxic, non-hemolytic and exhibit efficacy in mixed bacterial infection model which closely represents a chronic wound setting. Besides having strong anti-bacterial potential, the variant peptides also displayed wound healing functions, marking them as ideal therapeutics against chronic wound infections. Besides chronic wound healing, peptides also exhibited wider applicability to treat skin, soft tissue, lung, ear, and eye infections. None of the existing antimicrobial peptides (AMPs) exhibit these advantages despite years of research.

TABLE 13
Comparison of AMPs in clinical trial with peptide V8LQ19L

| | | | | | | | Broad | | | |
|---|---|---|---|---|---|---|---|---|---|---|
| | | Clinical | | | | [symbol] | spectrum | Healing | | |
| | AMP [symbol] | trial | Application | [symbol] | Size | prevention | activity | support | [symbol] | [symbol] |
| | [symbol] | [symbol] | [symbol] | [symbol] | [symbol] | ✓ | X | ✓ | ✓ | X |
| | [symbol] | [symbol] | [symbol] | [symbol] | [symbol] | X | X | ✓ | ✓ | X |
| | [symbol] | [symbol] | [symbol] | [symbol] | [symbol] | ✓ | ✓ | ✓ | X | ✓ |
| | [symbol] | [symbol] | [symbol] | [symbol] | [symbol] | ✓ | ✓ | ✓ | ✓ | X |
| | [symbol] | [symbol] | [symbol] | [symbol] | [symbol] | [symbol] | X | X | ✓ | Not |
| | | | | | | | | | | reported |
| | [symbol] | [symbol] | [symbol] | [symbol] | [symbol] | ✓ | X | ✓ | ✓ | ✓ |
| | [symbol] | [symbol] | [symbol] | [symbol] | 23 | ✓ | X | ✓ | ✓ | ✓ |
| | [symbol] | [symbol] | [symbol] | [symbol] | [symbol] | [symbol] | X | Not | ✓ | Not |
| | | | | | | | | reported | | reported |
| | [symbol] | [symbol] | [symbol] | [symbol] | [symbol] | Not | X | ✓ | ✓ | ✓ |
| | | | | | | reported | | | | |
| SEQ ID | [symbol] | [symbol] | [symbol] | [symbol] | 22 | X | ✓ | ✓ | ✓ | ✓ |
| NO: 54 | | | | | | ( [symbol] ) | | | | |
| SEQ ID | V8LQ19L | | [symbol] | [symbol] | [symbol] | ✓ | ✓ | ✓ | ✓ | ✓ |
| NO: 39 | | | | | | | | | | |

[symbol]

indicates data missing or illegible when filed

Active peptide sequences based on ENO3 exerting significant bactericidal antibiofilm properties, and relevant to chronic wound healing are previously disclosed. The previously disclosed antimicrobial peptides (AMPs) are longer (>22-residues) in length, which would be more costly in production. Comparative data between the wild-type ENO3-based peptides (EIA) and the variant peptides described herein shows advantageous effects of the variant peptides (Table 14).

TABLE 14
Comparison of wild-type peptides to variant peptides

| | Wild-type peptide E1A | Peptide V8LQ19L |
|---|---|---|
| Characteristics | (SEQ ID NO: 1) | (SEQ ID NO: 39) |
| Killing efficacy | Does not achieve >99% | >99% (MIC/MBC = 1.56 μM) |
| | inhibition or killing, even at | |
| | 500 μM, indicating the | |
| | MIC/MBC cannot be | |
| | determined | |
| Cationicity | Non-cationic (cannot disrupt | Cationic (bacterial membrane |
| | bacterial membrane) | disruptor) |
| Mechanism of killing bacteria | Unknown/non-membrane | Membrane disintegration |
| | disruptor | |
| Termini-modification | No | C-termini amidated |

Additionally, Table 15 below provides comparison between the variant peptides as described herein and known potent antimicrobial peptide (AMP) Pexiganan.

TABLE 15
Comparison of peptide V8LQ19L to Pexiganan

| | Pexiganan | V8LQ19L |
|---|---|---|
| Characteristics | (SEQ ID NO: 54) | (SEQ ID NO: 39) |
| Size | 22-residues | ≤21-residues |
| AMR prevention in PA and SA | Gives rise to AMR | Prevent AMR |
| MIC/MBC against PA | 3.125/6.25 | 1.56/1.56 |
| MIC/MBC against SA | 6.12/12.5 | 1.56/1.56 |
| Cytotoxicity at high | Cytotoxic | Non-cytotoxic |
| concentration (100 μM) | | |

TABLE 16
Comparison of peptide V8LQ19L to Standard of care

| | | Silver/Iodine- | H₂O₂/Peroxide- | | | |
|---|---|---|---|---|---|---|
| Features | Antibiotics | based products | generating products | Granudacyn | Prontosan | Lead peptide |
| Antibacterial Efficacy | Limited/Narrow | Broad- | | No | Broad- | Broad- |
| | | spectrum | | | spectrum | spectrum |
| AMR Prevention | No | | | No reports available | | Yes |
| Wound Healing Support | Impairs wound healing | | | | | Yes |
| Negligible Cytotoxicity | Cytotoxicity observed | | | | | Yes |
| No Allergic/Adverse | Contact irritation | ROS-mediated | Skin damage | Yes | Irritation | Yes |
| Reactions | Allergic dermatitis | stress responses | Epiderital necrosis | | | |

| | | |
|---|---|---|
| Targeted bacteria- | No such potential | Yes |
| specific eradication | | |

TABLE 17
Bacterial strains according to their severity levels as determined by the WHO
The WHO Priority list for R&D of new antibiotics

| PRIORITY 1: CRITICAL | PRIORITY 2: HIGH | PRIORITY 3: MEDIUM |
|---|---|---|
| Acinetobacter baumannii | Enterococcus faecium | Streptococcus pneumoniae |
| Carbapenem resistant | vancomycin-resistant | penicillin-non-susceptible |
| Pseudomonas aeruginosa | Staphylococcus aureus | Haemophilus influenzae |
| Carbapenem resistant | methicillin-resistant | ampicillin-resistant |
| Enterobacteriaceae | vancomycin-intermediate | Shigella spp. |
| Carbapenem resistant, | and resistant | fluoroquinolone-resistant |
| ESBL-producing | Helicobacter pylori | |
| | clarithromycin-resistant | |
| | Campylobacter spp. | |
| | fluoroquinolone-resistant | |
| | Salmonellae | |
| | fluoroquinolone-resistant | |
| | Neisseria gonorrhoeae | |
| | cephalosporin-resistant | |
| | fluoroquinolone-resistant | |

The discovery of the variant peptides described herein is not novel to a person skilled in the art. The variant peptides comprise a serine and two alanine residues in their conserved sequences, which are required for good anti-bacterial activity. These two amino acids are not known to enhance anti-bacterial activity and would not have been thought to be included into a peptide sequence. Although it is possible to predict the effect of modifications via structural analysis, peptides behave differently in practice. Thus, mere prediction based on bioinformatics would not be sufficient and should be tested with real experimental data, which poses undue burden to person skilled in the art.

### Sequences

The sequences disclosed in the present application include:

| Peptides | Sequence | SEQ ID NOS |
|---|---|---|
| E1AM1-NH2 | KKNQKKSVKLSKKALKLAKKN | 1 |
| E1AM2-NH2 | KKLLKKWVKLSKKALKLAQSL | 2 |
| Ac-E1AM2 | Ac- KKLLKKWVKLSKKALKLAQSL | 3 |
| Ac-E1AM2-NH2 | Ac- KKLLKKWVKLSKKALKLAQSL | 4 |
| E1AM2 N-1-NH2 | KLLKKWVKLSKKALKLAQSL | 5 |
| E1AM2 N-2-NH2 | LLKKWVKLSKKALKLAQSL | 6 |
| E1AM2 N-3-NH2 | LKKWVKLSKKALKLAQSL | 7 |
| E1AM2 N-4-NH2 | KKWVKLSKKALKLAQSL | 8 |
| E1AM2 N-5-NH2 | KWVKLSKKALKLAQSL | 9 |
| E1AM2 N-6-NH2 | WVKLSKKALKLAQSL | 10 |
| E1AM2 C-1-NH2 | KKLLKKWVKLSKKALKLAQS | 11 |
| E1AM2 C-2-NH2 | KKLLKKWVKLSKKALKLAQ | 12 |
| E1AM2 C-3-NH2 | KKLLKKWVKLSKKALKLA | 13 |
| E1AM2 C-4-NH2 | KKLLKKWVKLSKKALKL | 14 |
| E1AM2 C-5-NH2 | KKLLKKWVKLSKKALK | 15 |
| E1AM2 C-6-NH2 | KKLLKKWVKLSKKAL | 16 |
| E1AM2 W7L-NH2 | KKLLKKLVKLSKKALKLAQSL | 17 |
| E1AM2 W7K-NH2 | KKLLKKKVKLSKKALKLAQSL | 18 |
| E1AM2 V8L-NH2 | KKLLKKWLKLSKKALKLAQSL | 19 |
| E1AM2 V8K-NH2 | KKLLKKWKKLSKKALKLAQSL | 20 |
| E1AM2 S11L-NH2 | KKLLKKWVKLLKKALKLAQSL | 21 |
| E1AM2 S11K-NH2 | KKLLKKWVKLKKKALKLAQSL | 22 |
| E1AM2 A14L-NH2 | KKLLKKWVKLSKKLLKLAQSL | 23 |
| E1AM2 A14K- | KKLLKKWVKLSKKKLKLAQSL | 24 |
| NH2 | | |
| E1AM2 A18L-NH2 | KKLLKKWVKLSKKALKLLQSL | 25 |
| E1AM2 A18K- | KKLLKKWVKLSKKALKLKQSL | 26 |
| NH2 | | |
| E1AM2 Q19L- | KKLLKKWVKLSKKALKLALSL | 27 |
| NH2 | | |
| E1AM2 Q19K- | KKLLKKWVKLSKKALKLAKSL | 28 |
| NH2 | | |
| E1AM2 S20L-NH2 | KKLLKKWVKLSKKALKLAQLL | 29 |
| E1AM2 S20K-NH2 | KKLLKKWVKLSKKALKLAQKL | 30 |
| E1AM2 V8L Q19L | KKLLKKWLKLSKK(Aib)LKLALSL | 31 |
| A14Aib-NH2 | | |
| E1AM2 V8L Q19L | KKLLKKWLKLSKKALKL(Aib)LSL | 32 |
| A18Aib-NH2 | | |
| E1AM2 V8L Q19L | KKLLKKWLKLSKK(Aib)LKL(Aib)LSL | 33 |
| A1418Aib-NH2 | | |
| E1AM2 W7L V8L | KKLLKKLLKLSKKALKLALSL | 34 |
| Q19L-NH2 | | |
| E1AM2 W7(Bip) | KKLLKK(Bip)LKLSKKALKLALSL | 35 |
| V8L Q19L-NH2 | | |
| E1AM2 W7(1-Nal) | KKLLKK(1-Nal)LKLSKKALKLALSL | 36 |
| V8L Q19L-NH2 | | |
| E1AM2 W7(2-Nal) | KKLLKK(2-Nal)LKLSKKALKLALSL | 37 |
| V8L Q19L-NH2 | | |
| E1AM2 D-V8L | kkllkkwlklskkalklalsl | 38 |
| Q19L-NH2 | | |
| E1AM2 V8L | KKLLKKWLKLSKKALKLALSL | 39 |
| Q19L-NH2 | | |
| E1AM2 V8L Q19L | KLLKKWLKLSKKALKLALSL | 40 |
| N-1-NH2 | | |
| E1AM2 V8L Q19L | LLKKWLKLSKKALKLALSL | 41 |
| N-2-NH2 | | |
| E1AM2 V8L Q19L | LKKWLKLSKKALKLALSL | 42 |
| N-3-NH2 | | |
| E1AM2 V8L Q19L | KKLLKKWLKLSKKALKLALS | 43 |
| C-1-NH2 | | |
| E1AM2 V8L Q19L | KKLLKKWLKLSKKALKLAL | 44 |
| C-2-NH2 | | |
| E1AM2 V8L Q19L | KKLLKKWLKLSKKALKLA | 45 |
| C-3-NH2 | | |
| E1AM2 D-V8L | kkllkkwlklskkalklal | 46 |
| Q19L C-2-NH2 | | |
| E1AM2 | KKLLKK(2-Nal)LKLSKKALKLAL | 47 |
| W7(2-Nal) V8L | | |
| Q19L C-2-NH2 | | |
| E1AM2 D- | kkllkk(2-nal)lklskkalklal | 48 |
| W7(2-Nal) V8L | | |
| Q19L C-2-NH2 | | |
| General formula I | KKLLKKX₁X₂KLX₃KKX₄L | 49 |
| General formula II | X₁X₂KLX₃KKX₄LKLX₅X₆X₇L | 50 |
| General formula III | KKLLKKX₁X₂KLX₃KKX₄LKLX₅X₆X₇L | 51 |
| Human ENO3 | MAMQKIFAREILDSRGNPTVEVDLHTAKGRFR | 52 |
| | AAVPSGASTGIYEALELRDGDKGRYLGKGVLK | |
| | AVENINNTLGPALLQKKLSVVDQEKVDKFMIEL | |
| | DGTENKSKFGANAILGVSLAVCKAGAAEKGVP | |
| | LYRHIADLAGNPDLILPVPAFNVINGGSHAGNK | |
| | LAMQEFMILPVGASSFKEAMRIGAEVYHHLKG | |
| | VIKAKYGKDATNVGDEGGFAPNILENNEALELL | |
| | KTAIQAAGYPDKVVIGMDVAASEFYRNGKYDL | |
| | DFKSPDDPARHITGEKLGELYKSFIKNYPVVSIE | |
| | DPFDQDDWATWTSFLSGVNIQIVGDDLTVTNPK | |
| | RIAQAVEKKACNCLLLKVNQIGSVTESIQACKL | |
| | AQSNGWGVMVSHRSGETEDTFIADLVVGLCTG | |
| | QIKTGAPCRSERLAKYNQLMRIEEALGDKAIFA | |
| | GRKFRNPKAK | |
| E1A | KVNQIGSVTESIQACKLAQSN | 53 |
| Pexiganan | GIGKFLKKAKKFGKAFVKILKK | 54 |

1. A peptide comprising the sequence of KKLLKKX1X2KLX3KKX4L (SEQID NO: 49), wherein:
X1 is S, W, L, K, 4-biphenyl-L-alanine, 1-naphthyl-L-alanine, or 2-naphthyl-L-alanine;
X2 is V, L, or K;
X3 is S, L, or K; and
X4 is A, L, K, or 2-aminoisobutyric acid.

2. The anti-bacterial peptide of claim 1, wherein the peptide sequence comprises the sequence KKLLKKWVKLSKKAL (SEQ ID NO: 6).

3. The peptide of claim 1, wherein the peptide sequence consists of any one of the following:
(SEQ ID NO: 11)
(a) KKLLKKWVKLSKKALKLAQS;
(SEQ ID NO: 12)
(b) KKLLKKWVKLSKKALKLAQ;
(SEQ ID NO: 13)
(c) KKLLKKWVKLSKKALKLA;
(SEQ ID NO: 14)
(d) KKLLKKWVKLSKKALKL;
(SEQ ID NO: 15)
(e) KKLLKKWVKLSKKALK;
(SEQ ID NO: 16)
(f) KKLLKKWVKLSKKAL;
or
(SEQ ID NO: 1)
(g) KKNQKKSVKLSKKALKLAKKN

4. A peptide comprising the sequence of X1X2KLX3KKX4LKLX5X6X7L (SEQ ID NO: 50), wherein:
X1 is S, W, L, K, 4-biphenyl-L-alalnine, 1-naphthyl-L-alanine, or 2-naphthyl-L-alanine;
X2 is V, L, or K;
X3 is S, L, or K;
X4 is A, L, K, or 2-Aminoisobutyric acid;
X5 is A, L, K, or 2-Aminoisobutyric acid;
X6 is Q, L, or K; and
X7 is S, L, or K.

5. The peptide of claim 4, wherein the peptide sequence comprises the sequence of WVKLSKKALKLAQSL (SEQ ID NO: 10).

6. The peptide of claim 4, wherein the peptide sequence consists of any one of the following
(SEQ ID NO: 5)
(a) KLLKKWVKLSKKALKLAQSL;
(SEQ ID NO: 6)
(b) LLKKWVKLSKKALKLAQSL;
(SEQ ID NO: 7)
(c) LKKWVKLSKKALKLAQSL;
(SEQ ID NO: 8)
(d) KKWVKLSKKALKLAQSL;
(SEQ ID NO: 9)
(e) KWVKLSKKALKLAQSL;
or
(SEQ ID NO: 10)
(f) WVKLSKKALKLAQSL

7. A peptide comprising the sequence of KKLLKKX1X2KLX3KKX4LKLX5X6X7L (SEQ ID NO: 51), wherein:
X1 is S, W, L, K, 4-biphenyl-L-alalnine, 1-naphthyl-L-alanine, or 2-naphthyl-L-alanine;
X2 is V, L, or K;
X3 is S, L, or K;
X4 is A, L, K, or 2-aminoisobutyric acid;
X5 is A, L, K, or 2-aminoisobutyric acid;
X6 is Q, L, or K; and
X7 is S, L, or K.

8. The peptide of claim 7, wherein the peptide sequence consists of any one of the sequences of
(E1AM2, SEQ ID NO: 2)
(i) KKLLKKWVKLSKKALKLAQSL;
(E1AM2 Q19L, SEQ ID NO: 27)
(ii) KKLLKKWVKLSKKALKLALSL;
(E1AM2 V8L, SEQ ID NO: 19)
(iii) KKLLKKWLKLSKKALKLAQSL;
(E1AM2 V8L Q19L, SEQ ID NO: 39)
(iv) KKLLKKWLKLSKKALKLALSL;
(E1AM2 V8L Q19L C-2, SEQ ID NO: 44)
(v) KKLLKKWLKLSKKALKLALSL;
or
(E1AM2 W7(2-Nal) V8L Q19L C-2, SEQ ID NO: 47)
(vi) KKLLKK(2-Nal)LKLSKKALKLAL.

9. The peptide of claim 1, wherein the peptide further comprises a partial sequence from ENO3.

10. The peptide of claim 1, wherein the peptide has less than 22 amino acid residues.

11. A composition comprising one or more of the peptides of claim 1.

12. A pharmaceutical composition comprising the peptide of claim 1 and a pharmaceutically acceptable carrier or excipient.

13. A wound dressing comprising the peptide of claim 1.

14. A nucleic acid composition encoding the peptide of claim 1.

15. An expression vector comprising the nucleic acid composition of claim 14.

16. A host cell comprising at least one nucleic acid composition of claim 14.

17.-19. (canceled)

20. A pharmaceutical composition comprising the composition of claim 11 and a pharmaceutically acceptable carrier or excipient.

21. A wound dressing comprising the composition of claim 11.

22. A wound dressing comprising the pharmaceutical composition of claim 12.

23. A host cell comprising at least one expression vector of claim 15.
